SEQUENCE LISTING (18 sequences)

SEQ ID NO: 1 - protein, 21 aa, Homo sapiens
LKGQAFLPLVLEPRRLPVGPL

SEQ ID NO: 2 - protein, 16 aa, Homo sapiens
PLKGQAFLPLVLEPRR

SEQ ID NO: 3 - protein, 17 aa, Homo sapiens
PLKGQAFLPLVLEPRRL

SEQ ID NO: 4 - protein, 21 aa, Homo sapiens
SPEEPTPLPVLLLLPPPSTPA

SEQ ID NO: 5 - protein, 22 aa, Homo sapiens
VDYGKLPPAPALLAPYEVLGGA

SEQ ID NO: 6 - protein, 22 aa, Homo sapiens
SQPQFPAVPLELLPTTEGTPGL

SEQ ID NO: 7 - protein, 16 aa, Homo sapiens
KYTRLQTPSSFLTLAT

SEQ ID NO: 8 - protein, 21 aa, Homo sapiens
YLSRFGEVVDRTIKTDPVTGR

SEQ ID NO: 9 - protein, 22 aa, Homo sapiens
RKYVAAQLHLHCGQKGSPGGSE

SEQ ID NO: 10 - protein, 22 aa, Homo sapiens
PFLHTVSKTRLFEYLRLTSLGV

SEQ ID NO: 11 - protein, 22 aa, Homo sapiens
TGACPWSGSFLLENTELANVGA

SEQ ID NO: 12 - protein, 21 aa, Homo sapiens
NSEVVMETANCVLRNHSQRQG

SEQ ID NO: 13 - protein, 22 aa, Homo sapiens
YWPHEIPKVFDLEKERESGVLK

SEQ ID NO: 14 - protein, 15 aa, Homo sapiens
ELLLHPGALGAPGAG

SEQ ID NO: 15 - protein, 22 aa, Homo sapiens
RLVFRVHVPQSSGRTLSLQVAS

SEQ ID NO: 16 - protein, 21 aa, Homo sapiens
GARKISSEPVSGEIIAVRVKE

SEQ ID NO: 17 - protein, 22 aa, Homo sapiens
SKGKSPSRNKKSSLTAVIDKLK

SEQ ID NO: 18 - protein, 22 aa, Homo sapiens
GDTGAQGLPGPSGEDGERGDDG

(19) United States
(12) Patent Application Publication
(10) Pub. No.: US 2025/0197924 A1
(43) Pub. Date: Jun. 19, 2025

(54) METHODS FOR SELECTION AND COMBINATION OF SEQUENCING RESULTS FROM BIOLOGICAL SAMPLES FOR NEOANTIGEN SCORING

(71) Applicant: AMAZON TECHNOLOGIES, INC., RENO, NV (US)

(72) Inventors: Haibao TANG, Cupertino, CA (US); Anta IMATA SAFO, Seattle, WA (US); Alena HARLEY, Los Gatos, CA (US); Frank Wilhelm SCHMITZ, Seattle, WA (US); Antje HEIT, Seattle, WA (US); Layne Christopher PRICE, Seattle, WA (US); David HECKERMAN, Bellevue, WA (US); Samuel Anthony DANZIGER, Seattle, WA (US)

(21) Appl. No.: 18/542,383

(22) Filed: Dec. 15, 2023

(51) Int. Cl. C12Q 1/6869; C12Q 1/6886

(52) U.S. Cl. CPC C12Q 1/6869; C12Q 1/6886; C12Q 2600/106

(57) ABSTRACT

Disclosed herein are methods of scoring predicted immunogenicity of neoantigens from biological samples of a subject. Methods can include the steps of preparing biological samples for nucleic acid sequencing; nucleic acid sequencing; evaluating the initial sequencing results by analyzing (e.g., comparing) sequencing parameters of the results; based on an analysis (e.g., a comparison) of sequencing parameters, combining the initial sequencing results to yield union sequencing results or selecting a representative biological sample; and scoring the predicted immunogenicity of neoantigens in the biological samples based on either the union sequencing results or the sequencing results of the representative sample. Methods can further include the step of comparing sequencing parameters of union sequencing results and the initial sequencing results. Methods can further include the steps of generating a neoantigen vaccine that contains or encodes for a neoantigen scored for predicted immunogenicity and administering the neoantigen vaccine to a subject.

### BACKGROUND

Since the sequencing and assembly of the first human genome by the Human Genome Project in 2003, clinicians and scientists have envisioned the treatment of a range of diseases informed by personalized genetic diagnoses and therapeutics. Cancer, a disease that is predicted to kill an estimated 609,823 people in the U.S. in 2023 (see R. L. Siegel, et al. Cancer statistics, 2023. CA Cancer J. Clin. (2023) 73, 17-48), is particularly suitable for personalized genetic diagnoses and therapeutics based on genetic sequencing as cancers originate through somatic mutations over time in the genome of non-cancerous or precancerous cells in a patient (see E. D. Pleasance et al. A comprehensive catalogue of somatic mutations from a human cancer genome. Nature (2010), 463, 191-196; and M. R. Stratton et al. The cancer genome. Nature (2009), 458 (7239), 719-724).

Cancerous malignancies (e.g., solid tumors) are comprised of a heterogenous mixture of subclones derived from a common clonal cell. This is a result of selection, drift, and spatial separation of clonal populations within the tumor (see M. Tarabichi et al. A practical guide to cancer subclonal reconstruction from DNA sequencing Nat. Methods (2021), 18 (2), 144-155). The mutations present in different subclones are important for the diagnosis and treatment of cancer as they can confer resistance to chemotherapeutic agents or render personalized immunotherapeutic agents (e.g., CAR T cells, neoantigen vaccines) ineffective (see D. J. Craig, et al. Subclonal landscape of cancer drives resistance to immune therapy. Cancer Treat. Res. Commun. (2022), 30, 100507, 1-6).

Unfortunately, tumor heterogenicity poses a challenge for obtaining a biological sample (e.g., a tissue biopsy) that is representative of the genomic variants present in the whole tumor. Nucleic acids from biological samples are conventionally evaluated for heterogenicity of genomic variants by sequencing, incurring excessive costs and requiring expenditure of time on the exhaustive sequencing of samples that may be inevitably non-representative of the tumor and may not provide representative basis for informing patient treatment and/or personalized therapy development. Collection of further, more representative samples by additional procedures inconveniences the patient and poses an additional risk of infection or other surgical complications. Consequently, there is an unmet need for methods to efficiently obtain sequencing results representative of the tumor heterogenicity for ranking of predicted immunogenicity and selection of neoantigens for use in patient treatments (e.g., generation of a neoantigen vaccine, informing clinicians on the most efficacious treatment for a heterogenous cancerous malignancy in a patient).

The heterogenicity of tumor somatic mutations and the spatial separation of related but distinct subclonal populations within tumors make it difficult to ensure a biological sample (e.g., a tissue biopsy sample) is representative of the majority of the subclonal populations. Samples representative of the genomic variance of tumor subpopulations are important for the genomic analysis of a cancer as a whole, the diagnosis of driver mutations in a cancer, the selection of appropriate chemotherapeutic and immunotherapeutic agents for the treatment of a cancer, and the generation of personalized therapies (e.g., cell therapies, neoantigen vaccines). One solution to this problem is to collect multiple biological samples (e.g., tissue biopsy samples) from a patient (e.g., collected during a surgical procedure) in an effort to identify a sample representative of the tumor subpopulations. Unfortunately, identification and selection of a representative biological sample (e.g., tissue biopsy sample) by conventional methodologies requires expensive, time-consuming, and exhaustive sequencing of each candidate biological sample (e.g., tissue sample).

Described herein are methods of scoring (e.g., ranking, evaluating) predicted immunogenicity of neoantigens in one or more biological samples of a subject in need thereof. The methods can include the steps of preparing biological samples (e.g., two or more biological samples) for nucleic acid sequencing; nucleic acid sequencing of the biological samples to yield initial sequencing results for each of the biological samples; evaluating the initial sequencing results by analyzing (e.g., comparing) sequencing parameters (e.g., one or more sequencing parameters) of the initial sequencing results; based on an analysis (e.g., a comparison) of the sequencing parameters of the initial sequencing results of the biological samples, combining the initial sequencing results of the biological samples to yield union sequencing results; and scoring the predicted immunogenicity of neoantigens (e.g., one or more neoantigens) in the biological samples of the subject in need thereof based on the union sequencing results of the biological samples. The method can include the steps of preparing biological samples (e.g., two or more biological samples) for nucleic acid sequencing; nucleic acid sequencing of the biological samples to yield initial sequencing results for each of the biological samples; evaluating the initial sequencing results by analyzing (e.g., comparing) sequencing parameters of the initial sequencing results; based on an analysis (e.g., a comparison) of the sequencing parameters of the initial sequencing results of the biological samples, selecting a representative biological sample; and scoring the predicted immunogenicity of neoantigens in the representative sample of the subject in need thereof based on the initial sequencing result of the representative biological sample.

The biological samples used in methods described herein can be any biological sample from a subject. Each biological sample can independently be an excisional biopsy, a liquid biopsy, an incisional biopsy, a needle biopsy, a punch biopsy, or a shave biopsy. The biological samples can be from any region of the body of a subject (e.g., any region of the body, any region of a tumor, any region of an organ). At least two of the biological samples can be from different regions of the body of a subject. At least two of the biological samples can be from different regions of a tumor of a subject. Biological samples can be from any tissue or organ of a subject. The biological samples can be from any type of tumor. A biological sample (e.g., at least one of the biological samples) can be from a primary tumor of a subject. A biological sample (e.g., at least one of the biological samples) can be from a secondary (e.g., metastatic) tumor of a subject.

Biological samples can be collected at different times, with any duration of time between the collection of each sample. Biological samples (e.g., at least two of the biological samples) can be collected from a subject at different occasions that are temporally separated by at least about 1 day.

Sequencing parameters of sequencing results (e.g., union sequencing results, subsequent sequencing results, initial sequencing results) used in methods described herein can be any type of sequencing parameters. The sequencing parameters can be a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, or a combination of any of the foregoing.

Any type of sequencing results (e.g., initial sequencing results, union sequencing results, representative biological sample sequencing results) can be obtained and used in methods described herein. The sequencing results (e.g., the initial sequencing results) can be sequencing reads, sequencing variants, encoded peptides, or a combination of the foregoing.

Methods described herein can further include the step of comparing union sequencing parameters of the union sequencing results (e.g., the results obtained by combining initial sequencing results of biological samples) to the sequence parameters of the initial sequencing results.

The nucleic acid sequencing of methods described herein can be sequencing of any type of nucleic acid. The type of nucleic acid can be RNA, DNA, or a combination of the foregoing. The sequencing (e.g., nucleic acid sequencing, subsequent sequencing) of the method can be using any sequencing technique. The sequencing can be whole exome sequencing, whole genome sequencing, RNA sequencing, single cell sequencing, targeted panel sequencing, or a combination of the foregoing. Any number of different sequencing techniques can be used in the sequencing in the method (e.g., nucleic acid sequencing). At least two different sequencing techniques can be used in the sequencing steps of the method. Any number of biological samples (e.g., at least two of the biological samples) can be sequenced by different sequencing techniques, such as at the step of nucleic acid sequencing. Any sample (e.g., a biological sample, a representative biological sample, a biological sample combined to yield union sequencing results) can be submitted to subsequent sequencing.

The neoantigens can be any length of peptides. The neoantigen peptides can be long peptides, short peptides, or a combination of the foregoing. The method can further include the step of generating a neoantigen vaccine. The neoantigen vaccine can contain or encode for neoantigens scored for predicted immunogenicity by methods described herein. The method can further include the step of administering the neoantigen to a subject, such as a cancer patient.

### BRIEF DESCRIPTION OF DRAWINGS

FIG. 1 is a flow diagram showing steps of an exemplary method for scoring predicted immunogenicity of neoantigens as described herein. Tissue biopsies 1 and 2 (left hand portion of FIG. 1) are collected from a tumor and, following preparation for sequencing, are submitted for sequencing using a sequencing technique such as whole exome sequencing (WES), whole genome sequencing (WGS), RNA sequencing, or the like. Genomic variants (a-i, FIG. 1 at center) are identified in the sequencing results from each tissue biopsy sample. The sequencing results can be combined to yield union sequencing results (e.g., “Core Union,” lower right-hand portion of FIG. 1) or a representative tissue biopsy sample can be selected (e.g., “Core Selection”) for scoring of predicted immunogenicity of neoantigens in the sample. The combined samples or the selected samples can be submitted to subsequent sequencing by a sequencing technique (e.g., deeper WGS, middle right-hand portion of FIG. 1). Based on the sequence parameters of the initial sequencing results, the union sequencing results or the sequencing results of the selected representative sample can lead to improved coverage and/or depth of sequencing of variants for downstream applications such as neoantigen vaccine generation.

FIG. 2 is an exemplary result of scoring predicted immunogenicity of neoantigens. Experimental details yielding this result are presented in Example 1. Initial sequencing results from whole exome sequencing of two core needle biopsies (i.e., Core #1, Core #2) from a patient (i.e., patient A) each yielded a list of encoded neoantigen peptides (left and middle columns of FIG. 2). The sequencing results were combined to yield union sequencing results as a list of encoded neoantigen peptides (right column of FIG. 2). The score of predicted immunogenicity is shown to the left of each neoantigen peptide sequence. Each list is ranked by predicted immunogenicity score. Exemplary neoantigen peptides that overlap or encompass one another are denoted with a “*” or “#” symbol in each list.

FIGS. 3A and 3B are Venn diagrams of long identified neoantigen peptides from tissue core needle biopsies of two patients. FIG. 3A is a Venn diagram of long identified neoantigen peptides from the same core needle biopsies of a patient (i.e., patient A) as the results displayed in FIG. 2. The diagram shows that only one neoantigen peptide is exclusive to each initial sequencing results of the core biopsy samples (i.e., “Core 1”, “Core 2”) and the combination of sequencing results (i.e., “Core Union”). The combination of sequencing results represents the majority of long identified neoantigen peptides in both Core 1 and Core 2 tissue biopsy samples. FIG. 3B is a Venn diagram of long identified neoantigen peptides from core needle biopsies of a different patient (i.e., patient B) than the results displayed in FIG. 2. The diagram shows that six neoantigen peptides are exclusive to only one of the core needle biopsy samples (i.e., “Core 2”). Core 2 contains 55 out of 58 of the long identified neoantigen proteins. In this example, further sequencing of Core 2 could be conducted and Cores 1 and 2 may not be combined by methods described herein.

FIGS. 4A and 4B are Venn diagrams of neoantigen peptides selected for neoantigen vaccine generation from tissue core needle biopsies of two patients. FIG. 4A is a Venn diagram of a subset of neoantigen peptides selected for neoantigen vaccine generation from the same core needle biopsies of a patient (i.e., patient A) as the results displayed in FIG. 2 and FIG. 3A, based on the top 48 identified peptides by predicted immunogenicity. The diagram shows that several neoantigen peptides are exclusive for the biopsy samples (i.e., “Core 1”, “Core 2”) and the combination of sequencing results (i.e., “Core Union”). FIG. 4B is a Venn diagram of a subset of long identified neoantigen peptides from the same core needle biopsies of a patient (i.e., patient B) as the results displayed in FIG. 3B, based on the top 48 identified peptides by predicted immunogenicity. The diagram shows that several neoantigen peptides are exclusive to only one of the core needle biopsy samples (i.e., “Core 2”) and the combination of sequencing results (i.e., “Core Union”).

### DETAILED DESCRIPTION

The heterogenicity of tumor somatic mutations and the spatial separation of related but distinct subclonal populations within tumors make it difficult to ensure a biological sample (e.g., a tissue biopsy sample) is representative of the majority of the subclonal populations. Samples representative of the genomic variance of tumor subpopulations are important for the genomic analysis of a cancer as a whole, the diagnosis of driver mutations in a cancer, the selection of appropriate chemotherapeutic and immunotherapeutic agents for the treatment of a cancer, and the generation of personalized therapies (e.g., cell therapies, neoantigen vaccines). One solution to this problem is to collect multiple biological samples (e.g., tissue biopsy samples) from a patient (e.g., collected during a surgical procedure) in an effort to identify a sample representative of the tumor subpopulations. Unfortunately, identification and selection of a representative biological sample (e.g., tissue biopsy sample) by conventional methodologies requires expensive, time-consuming, and exhaustive sequencing of each candidate biological sample (e.g., tissue sample).

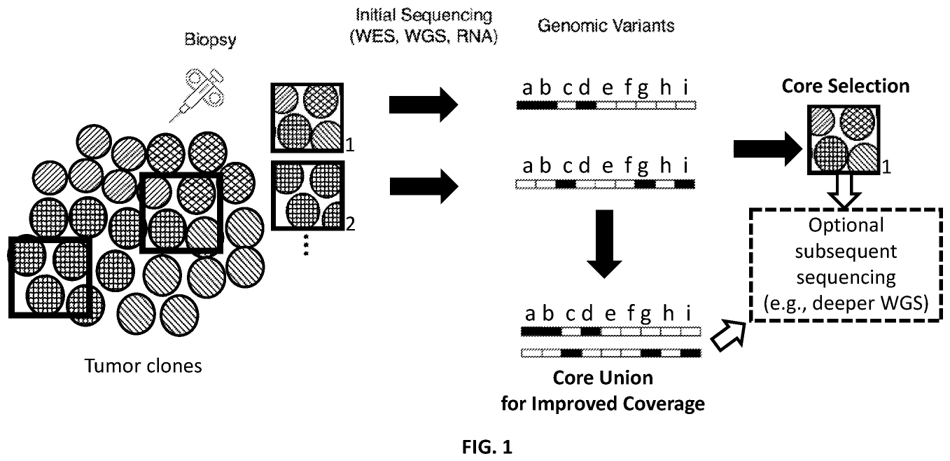

Described herein are methods of scoring (e.g., ranking, evaluating) predicted immunogenicity of neoantigens in one or more biological samples of a subject in need thereof. The method can include a n initial biological sample sequencing step for evaluating sequencing parameters of each biological sample (e.g., the quality of the sample, tumor purity, RNA quality, number of identified neoantigen peptides) (see FIG. 1). In one aspect, sequencing parameters of biological samples can be analyzed (e.g., compared) and based on this analysis (e.g., comparison), a biological sample can be selected as a representative biological sample. In such an aspect, based on the sequencing results of the representative biological sample, the methods described herein can score the predicted immunogenicity of neoantigens present in the representative biological sample. In another aspect, based on an analysis (e.g., comparison) of sequencing parameters of biological samples, the sequencing results of two or more biological samples can be combined to generate union sequencing results. In such an aspect, based on the union sequencing results of the two or more biological samples, the methods described herein can score the predicted immunogenicity of neoantigens present in a biological sample. It is believed, without wishing to be bound by any one particular theory, by initial sequencing of biological samples and analysis (e.g., comparison) of the sequencing parameters, a biological sample representative of a plurality (e.g., a majority) of subclones within a tumor can be selected or two or more biological sample sequencing results can be combined to form union sequencing results that are representative of a plurality (e.g., a majority) of subclones within a cancer. Advantageously, methods described herein can limit the cost and time associated with selecting a biological sample as an initial sequencing technique can be conducted on all collected samples and the initial sequencing technique can be limited to an initial shallow, targeted, and/or rapid sequencing technique. Selecting representative samples or combining two or more sequencing results by methods described herein yield representative sequencing results of tumor subclonal populations while reducing the time required, the data processing requirements, and costs compared to exhaustive sequencing of every biological sample collected.

Methods described herein can include the step of preparing biological samples (e.g., two or more biological samples) for nucleic acid sequencing. As described herein, any number of biological samples can be prepared (e.g., two or more, three or more, four or more, five or more, six or more, seven or more, eight or more, nine or more, ten or more) and any type of biological sample can be used including, but not limited to a biopsy sample (e.g., a liquid biopsy sample), surgically removed tissue sample, or a combination thereof. The step of preparing can include, but is not limited to weighing the biological sample, isolating nucleic acids (e.g., DNA, RNA) from the biological sample (e.g., isolating nucleic acids by any method including but not limited to organic extraction, solid phase extraction, Chelex extraction, DNA precipitation, or combinations thereof), measuring the amount of nucleic acid in the sample, adding RNase inhibitors, purifying the nucleic acids of the sample (e.g., by any method of nucleic acid purification, such as agarose gel extraction, column-based nucleic acid purification, or the like), determining the purity of the isolated nucleic acids, aliquoting the isolated nucleic acids, or combinations thereof.

Methods described herein can include the step of nucleic acid sequencing of biological samples (e.g., two or more biological samples) to yield initial sequencing results for each biological sample. As described herein, any nucleic acid sequencing technique can be used for nucleic acid sequencing or subsequent sequencing in the method. Suitable sequencing techniques include, but are not limited to whole genome sequencing, RNA sequencing, single cell sequencing (e.g., single cell RNA sequencing, single cell DNA sequencing, single cell whole exome sequencing, single cell whole genome sequencing), real-time PCR, deep sequencing, high-throughput sequencing (e.g., next generation sequencing), targeted panel sequencing, or combinations thereof. Methods can use any number (e.g., at least two) of different sequencing techniques at any step of the method. Methods can use the same sequencing technique for biological samples in any step of the method. Any type of nucleic acid can be sequenced in a step of the method (e.g., in a step of nucleic acid sequencing, in a step of subsequent sequencing) including, but not limited to DNA, RNA, or a combination thereof.

Methods described herein can include the step of evaluating initial sequencing results (e.g., the initial sequencing results of each biological sample) by analyzing (e.g., comparing) one or more sequencing parameters of the initial sequencing results. As described herein, the sequencing parameters of sequencing results (e.g., initial sequencing results, subsequent sequencing results, union sequencing results) can be any sequencing parameter. Suitable sequencing parameters include, but are not limited to number of sequence reads (e.g., number of total sequence reads), number of sequencing variants (e.g., number of DNA sequencing variants, number of RNA sequencing variants), tumor purity, sequencing depth, number of protein-modifying sequencing variants (e.g., number of protein-modifying sequencing variants confirmed by RNA sequencing), RNA confirmation rate, RNA quality, number of long identified neoantigen peptides, number of short identified neoantigen peptides, predicted immunogenicity of a neoantigen peptide, or combinations thereof. Any number of sequencing parameters can be analyzed or compared between sequencing results for biological samples or between a union sequencing result and an initial sequencing result of a biological sample. Without wishing to be bound by any one particular theory, it is believed that an analysis of sequencing parameters (e.g., a comparison of sequencing parameters between biological samples (e.g., between two or more biological samples)) can be used in determining whether to combine initial sequencing results of the two or more biological samples to yield union sequencing results or to select a representative biological sample. It is further believed that this methodology affords the most representative result of genomic variance (e.g., somatic mutations) present in the originating biological of the biological samples (e.g., a combination of samples, a selection of a representative sample) and that this representative result can be used to generate a treatment for a subject (e.g., generate a neoantigen vaccine).

Methods described herein can include the step of combining the initial sequencing results of two or more biological samples to yield union sequencing results, based on an analysis (e.g., a comparison) of sequencing parameters of the initial sequencing results of the biological samples. Methods described herein can include the step of selecting a representative biological sample, based on an analysis (e.g., a comparison) of sequencing parameters of the initial sequencing results of the biological samples. As described herein, the sequencing parameters of sequencing results (e.g., union sequencing results, initial sequencing results, subsequent sequencing results) can be analyzed (e.g., compared) by any mathematical calculation. One suitable mathematical calculation is a ratio between sequencing parameters of sequencing results. A ratio can indicate that sequencing results of two or more biological samples will be combined to form union sequencing results. A ratio can indicate that a biological sample (e.g., a single tissue sample) will be selected as a representative biological sample.

Methods described herein can include the step of scoring the predicted immunogenicity of neoantigens (e.g., one or more neoantigens in the biological samples) based on union sequencing results. Methods described herein can include the step of scoring the predicted immunogenicity of neoantigens (e.g., one or more neoantigens in the biological samples) based on a sequencing result of a representative biological sample. Any methodology for predicting immunogenicity of a neoantigen can be used in methods described herein.

In some embodiments, the method of scoring predicted immunogenicity of neoantigens in biological samples of a subject includes the steps of
a) preparing two or more biological samples for nucleic acid sequencing;
b) nucleic acid sequencing of the two or more biological samples to yield initial sequencing results for each of the biological samples;
c) evaluating the initial sequencing results by analyzing one or more sequencing parameters of the initial sequencing results;
d) based on an analysis of the sequencing parameters of the initial sequencing results of the biological samples, combining the initial sequencing results of the two or more biological samples to yield union sequencing results; and
e) scoring the predicted immunogenicity of one or more neoantigens in the biological samples of the subject in need thereof based on the union sequencing results of the biological samples.

In some embodiments, the method of scoring predicted immunogenicity of neoantigens in biological samples of a subject includes the steps of
a) preparing two or more biological samples for nucleic acid sequencing;
b) nucleic acid sequencing of the two or more biological samples to yield initial sequencing results for each of the biological samples;
c) evaluating the initial sequencing results by analyzing one or more sequencing parameters of the initial sequencing results;
d) based on an analysis of the sequencing parameters of the initial sequencing results of the biological samples, selecting a representative biological sample;
e) scoring the predicted immunogenicity of one or more neoantigens in the representative biological sample of the subject in need thereof based on the sequencing result of the representative biological sample.

Methods described herein can further include the step of comparing a union sequencing parameter of the union sequencing result to a sequence parameter of the initial sequencing results.

Methods described herein can further include the step of subsequent sequencing of a biological sample or a representative biological sample.

Methods described herein can further include the step of generating a neoantigen vaccine, wherein the neoantigen vaccine comprises or encodes for neoantigens scored for predicted immunogenicity by the method. The neoantigens can be peptides (e.g., long identified peptides, short identified peptides, synthesized peptides, isolated peptides), RNA sequences encoding for peptides (e.g., mRNA, mRNA containing unnatural nucleotides (e.g., pseudouridine, N1-methylpseudouridine, 7-methylguanosine, N6-methyladenosine, 2′-O-methyl nucleotide), mRNA containing inverted nucleotides, or combinations thereof), DNA sequences encoding for peptides (e.g., plasmid DNA), or combinations thereof.

Methods described herein can further include the step of administering the neoantigen vaccine to a subject in need thereof. The neoantigen vaccines can be administered to a subject that has been diagnosed with cancer, is already suffering from cancer, has recurrent cancer (i.e., relapse), or is at risk of developing cancer.

Methods described herein can be practiced on a patient (e.g., a human patient) as the subject. The patient can be suffering from any disorder or disease, such as cancer, infection, or genetic disorders. The methods described herein can be repeated any number of times during the treatment of a patient. The number of times a method can be repeated can be about 1, about 2, about 3, about 4, about 5, about 6, about 7, about 8, about 9, about 10, about 11, about 12, about 13, about 14, or about 15. As described herein, any duration of time can separate collection of samples and repetitions of the method.

### Definitions

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Unless otherwise indicated, the terms “at least,” “less than,” “about,” and “at most,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the singular forms “a,” “an,” and “the” include plural forms unless the context clearly indicates otherwise. The terms “include,” “such as,” and the like are intended to convey inclusion without limitation, unless otherwise specifically indicated.

As used herein, the term “cancer” refers to the physiological condition in subjects in which a population of cells is characterized by uncontrolled proliferation, immortality, metastatic potential, rapid growth and proliferation rate, and/or certain morphological features. Often cancers can be in the form of a tumor or mass, but may exist alone within the subject, or may circulate in the blood stream as independent cells, such as lymphoma cells. The term cancer includes all types of cancers and metastases, including hematological malignancy, solid tumors, sarcomas, carcinomas, and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu. Hormone receptor positive breast cancer refers to breast cancer that is positive for at least one of the following: ER or PR, and negative for Her2/neu (HER2).

The term “subject” as used herein refers to any animal, such as any mammal, including but not limited to, humans, non-human primates, dogs, cats, rodents (e.g., rats, mice, guinea pig, hamsters), and the like. In some embodiments, the mammal is a mouse. In some embodiments, the mammal is a human.

Any terms not directly defined herein shall be understood to have the meaning commonly associated with them as understood within the art of the invention. Any terms not directly defined herein shall be understood to have the meanings commonly associated with them as understood within the art of the invention. Certain terms are discussed herein to provide additional guidance to the practitioner in describing the compositions, devices, methods, and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Biological Samples

Biological samples used in methods of this disclosure can be obtained using any methodology and contain tissue or fluid from any bodily site, organ, or of any tissue type. A biological sample can be a biopsy sample (e.g., a tissue biopsy sample). A biopsy sample can be any type of biopsy including, but not limited to, an excisional biopsy, a liquid biopsy, an incisional biopsy, a needle biopsy, a punch biopsy, or a shave biopsy. A needle biopsy can be any type of needle biopsy including but not limited to, a core needle biopsy, a fine-needle aspiration biopsy, or a combination thereof. A needle biopsy can have any needle gauge including, but not limited to, 9, 10, 11, 12, 13, 14, 15, 16, 17, 18, 19, 20, 21, 22, 23, 24, 25, 26, 27, 28, 29, or 30. The needle biopsy gauge can range from 9 to 18, 10 to 17, 11 to 16, 12 to 15, 13 to 14, 20 to 30, 21 to 30, 22 to 30, 23 to 30, 9 to 30, 9 to 29, 9 to 28, 9 to 27, 9 to 26, 9 to 25, 9 to 24, 9 to 23, 9 to 22, 9 to 21, 9 to 20, 9 to 19, 9 to 17, 9 to 16, 9 to 15, 9 to 14, 9 to 13, 9 to 12, 9 to 11, 9 to 10, 10 to 30, 11 to 30, 12 to 30, 13 to 30, 14 to 30, 15 to 30, 16 to 30, 17 to 30, 18 to 30, or 19 to 30.

A biological sample of a subject can be a liquid biopsy. The liquid biopsy can be any biological fluid including, but not limited to an amniotic fluid sample, a blood sample, a buccal sample, a cerebrospinal fluid sample, a fecal sample, a peritoneal fluid sample, a pleural effusion sample (e.g., a transudative pleural effusion sample, an exudative effusion sample), a saliva sample, a semen sample, a synovial fluid sample, a urine sample, or a combination thereof. In contrast to a tissue biopsy, which comprises primarily solid biological tissue and is collected through a surgical procedure, a liquid biopsy can be collected from a vein or artery of a subject. The liquid biopsy can contain blood, circulating tumor cells, circulating tumor material (e.g., circulating tumor DNA (ctDNA), circulating tumor extracellular vesicles, circulating tumor proteins, circulating tumor RNA), plasma, serum, healthy blood cells, or a combination thereof. A liquid biopsy can be taken from a subject suffering from any type of disorder or condition including, but not limited to, an infection and a cancer (e.g., a hematological cancer, a solid tumor).

Biological samples can include two or more different biological sample types (e.g., two or more different biopsy types). For example, biological samples used in methods described herein can include three biopsy samples: two liquid biopsy samples and one tissue needle biopsy sample. Biological samples can include two or more of the same biopsy types. For example, the biological sample can include three tissue biopsy samples that are core needle biopsies. The number of different biological sample types can be 1, 2, 3, 4, 5, 6, 7, 8, 9, 10, 11, 12, 13, 14, 15, 16, 17, 18, 19, or 20. The number of biological sample types can be at least 2, at least 3, at least 4, at least 5, at least 6, at least 7, at least 8, at least 9, or at least 10. Sequencing parameters of sequencing results of two or more different biological sample types can be analyzed (e.g., compared) by methods described herein. The sequencing results of two or more different biological sample types can be combined by methods described herein to yield union sequencing results.

A biological sample (e.g. a tissue sample) can be any surgically removed tissue from a subject including, but not limited to, an adenectomy sample, adenoidectomy, adrenalectomy sample, apicoectomy sample, appendectomy sample, auriculectomy sample, bullectomy sample, bunionectomy sample, cecectomy sample, cervicectomy sample, cholecystectomy sample, colectomy sample, craniectomy sample, cystectomy sample, corpectomy sample, discectomy sample, diverticulectomy sample, duodenectomy sample, esophagectomy sample, extrapleural pneumonectomy sample, frenectomy sample, fundectomy sample, ganglionectomy sample, gastrectomy sample, gingivectomy sample, glossectomy sample, gonadectomy sample, hemicolectomy sample, hemilaminectomy sample, hemipelvectomy sample, hemispherectomy sample, hemorrhoidectomy sample, hepatectomy sample, hypophysectomy sample, hysterectomy sample, iridectomy sample, jejunectomy sample, keratectomy sample, laminectomy sample, laryngectomy sample, lumpectomy sample, lymphadenectomy sample, mastectomy sample, mastoidectomy sample, myocardiectomy sample, myomectomy sample, necrosectomy sample, nephrectomy sample, neurectomy sample, oophorectomy sample, orchiectomy sample, ostectomy sample, pancreatectomy sample, pancreaticoduodenectomy sample, panniculectomy sample, parathyroidectomy sample, pericardiectomy sample, pinealectomy sample, pneumonectomy sample, proctocolectomy sample, prostatectomy sample, pulpectomy sample, quadrantectomy sample, rhinectomy sample, salpingectomy sample, salpingo-oophorectomy sample, septectomy sample, splenectomy sample, stapedectomy sample, sympathectomy sample, thymectomy sample, thyroidectomy sample, tonsillectomy sample, trabeculectomy sample, tumor sample (e.g., a tumorectomy, a surgically excised tumor), turbinectomy sample, tubectomy sample, uterectomy sample, uvulectomy sample, vitrectomy sample, or combinations thereof. In some embodiments, the biological sample is a resection sample of a tumor.

A biological sample (e.g., a tissue biopsy, a surgically removed tissue) can be from any anatomical position. Anatomical positions include, but are not limited to, abdomen, ankle, arm, back, brachium, breast, buttocks, calf, chest, ear, elbow, eye, face, finger, foot, forearm, genitalia, hand, head, hip, knee, leg, mouth, neck, nose, scalp, shin, shoulder, thigh, toe, waist, and wrist. A biological sample can be from any organ including, but not limited to, adrenal glands, appendix, anus, artery, bladder, bone marrow, brain, bronchi, bronchioles, capillary, cervix, colon, ear, epididymis, esophagus, eye, fallopian tube, gallbladder, gut-associated lymphoid tissue, heart, interstitium, joint, kidney, large intestine, larynx, ligament, liver, lung, lymph node, mammary gland, mesentery, mouth, muscle, nasal cavity, nerve, olfactory epithelium, ovary, pancreas, pharynx, pineal gland, pituitary gland, placenta, prostate, rectum, salivary gland, skeleton, skin, small intestine, spinal cord, spleen, stomach, subcutaneous tissue, tendon, testicle, thymus, thyroid gland, tongue, trachea, ureter, urethra, uterus, vas deferens, ventricular system, and vein. The biological sample (e.g., tissue biopsy sample, a surgically removed tissue) can contain any bodily tissue including, but not limited to, connective tissue, epithelial tissue, muscular tissue, nervous tissue, or combinations thereof.

Methods described herein can be practiced on two or more biological samples from a subject. The number of biological samples can be 2, 3, 4, 5, 6, 7, 8, 9, 10, 11, 12, 13, 14, 15, 16, 17, 18, 19, or 20. The number of biological samples can be at least about 2, at least about 3, at least about 4, at least about 5, at least about 6, at least about 7, at least about 8, at least about 9, at least about 10, at least about 11, at least about 12, at least about 13, at least about 14, or at least about 15. Biological samples from a subject can be from the same region of the body of a subject. For example, three tissue samples could be from the left lung of a subject. Biological samples can be from different regions of the body of a subject. For example, a first tissue sample could be from the pancreas, and a second tissue sample could be from the bile duct of a subject. The number of biological samples from different regions of the body of a subject can be at least two, at least three, at least four, at least five, at least six, at least seven, at least eight, at least nine, or at least ten. Biological samples can be from the same region of a tumor of a subject. Biological samples can be from different regions of a tumor of a subject. For example, two tissue samples can include a first tissue sample that is collected from the posterior of an adenocarcinoma tumor and the second tissue sample is collected from the anterior of the same adenocarcinoma tumor. The number of biological samples from different regions of a tumor of a subject can be at least two, at least three, at least four, at least five, at least six, at least seven, at least eight, at least nine, or at least ten. Biological samples from a subject can be from a primary tumor of the subject. Biological samples from a subject can be from a secondary (e.g., metastatic tumor) of the subject. Two or more biological samples (e.g., tissue samples) can be from a different type of tumor including a primary tumor and a secondary tumor. For example, two tissue samples (e.g., a first tissue sample and a second tissue sample) could be collected and include a first tissue sample from a primary tumor from the liver and a second tissue sample could be from a metastatic tumor in the brain of a subject. In some embodiments, at least two of the biological samples are from different regions of the body of a subject. In some embodiments, at least two of the biological samples are from different regions of a tumor of a subject. In some embodiments, at least two of the biological samples are from different regions of a tumor of a subject.

The biological sample (e.g., the tissue biopsy sample, the surgically removed tissue, the liquid biopsy sample), can be collected and/or maintained at any temperature. For example, the biological sample can be collected and/or maintained at room temperature. For example, the biological sample can be collected and/or maintained frozen. The biological sample can be collected and/or maintained at about −78° C., about −76° C., about −74° C., about −72° C., about −70° C., about −68° C., about −66° C., about −64° C., about −62° C., about −60° C., about −58° C., about −56° C., about −54° C., about −52° C., about −50° C., about −48° C., about −46° C., about −44° C., about −42° C., about −40° C., about −38° C., about −36° C., −about 34° C., about −32° C., about −30° C., about −28° C., about −26° C., about −24° C., about −22° C., about −20° C., about −18° C., about −16° C., about −14° C., about −12° C., about −10° C., about −8° C., about −6° C., about −4° C., about −2° C., about 0° C., about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., or about 34° C. The biological sample can be collected and/or maintained at a temperature that is at most about −78° C., at most about −76° C., at most about −74° C., at most about −72° C., at most about −70° C., at most about −68° C., at most about −66° C., at most about −64° C., at most about −62° C., at most about −60° C., at most about −58° C., at most about −56° C., at most about −54° C., at most about −52° C., at most about −50° C., at most about −48° C., at most about −46° C., at most about −44° C., at most about −42° C., at most about −40° C., at most about −38° C., at most about −36° C., at most about −34° C., at most about −32° C., at most about −30° C., at most about −28° C., at most about −26° C., at most about −24° C., at most about −22° C., at most about −20° C., at most about −18° C., at most about −16° C., at most about −14° C., at most about −12° C., at most about −10° C., at most about −8° C., at most about −6° C., at most about −4° C., at most about −2° C., at most about 0° C., at most about 2° C., at most about 4° C., at most about 6° C., at most about 8° C., at most about 10° C., at most about 12° C., at most about 14° C., at most about 16° C., at most about 18° C., at most about 20° C., at most about 22° C., at most about 24° C., at most about 26° C., at most about 28° C., at most about 30° C., at most about 32° C., or at most about 34° C. The biological sample can be collected and/or maintained at a temperature range between about −78° C. to about −20° C., about −78° C. to about 0° C., about −78° C. to about 4° C., about −78° C. to about 24° C., about −20° C. to about 0° C., about −20° C. to about 4° C., about −20° C. to about 24° C., about 0° C. to about 4° C., about 0° C. to about 24° C., or about 4° C. to about 24° C.

Biological samples (e.g., tissues samples, tissue biopsy samples, surgically removed tissue samples) used in methods described herein can be from a tumor of a subject. The tumor can be any type of cancerous tumor, including hematological malignancies, solid tumors, sarcomas, carcinomas, and other solid and non-solid tumors. Illustrative suitable cancers include, for example, adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemias (e.g., acute lymphoblastic leukemia, acute myeloid leukemia, chronic lymphocytic leukemia, chronic myelogenous leukemia, hairy cell leukemia, myelodysplastic syndrome, prolymphocytic leukemia, large granular lymphocytic leukemia, adult T-cell leukemia, clonal eosinophilias), lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

Biological samples (e.g., tissue biopsy samples) used in methods described herein can be from primary tumors and/or secondary (e.g., metastatic) tumors of a subject. Any number of biological samples (e.g., of the two or more biological samples) can be from a primary tumor, such as at least 1, at least 2, at least 3, at least 4, at least 5, at least 6 at least 7, at least 8, at least 9, or at least 10. Any number of biological samples (e.g., of the two or more biological samples) can be from a secondary (e.g., a metastatic) tumor, such as at least 1, at least 2, at least 3, at least 4, at least 5, at least 6 at least 7, at least 8, at least 9, or at least 10. The tumor can be any stage of cancer including, but not limited to, stage 0, stage I, stage II, stage III, or stage IV. The tumor can be in remission (e.g., partial remission). The tumor can be a relapsed tumor (e.g., a tumor of a relapsed cancer). The tumor can have any grade including, but not limited to, X, 1, 2, 3, or 4. The tumor can be a recalcitrant tumor. The tumor can be resistant to therapy (e.g., resistant to chemotherapy, resistant to immunotherapy). The tumor can be susceptible to therapy (e.g., susceptible to chemotherapy, susceptible to immunotherapy, susceptible to radiotherapy). The tumor can be benign. The tumor can be precancerous. In some embodiments, at least one of the biological samples is from a primary tumor of the subject. In some embodiments, at least one of the biological samples is from a secondary tumor of the subject.

Biological samples in methods described herein can be collected at any time point in relation to each other. Biological samples (e.g., two or more biological samples) can be collected at the same time (e.g., during the same surgical procedure, on the same day). Biological samples can be collected from a subject at different occasions that are temporally separated by any duration of time. The duration of time between collection of two biological samples can be about 1 minute, about 2 minutes, about 3 minutes, about 4 minutes, about 5 minutes, about 6 minutes, about 7 minutes, about 8 minutes, about 9 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 1 day, about 2 days, about 3 days, about 4 days, about 5 days, about 6 days, about 7 days, about 8 days, about 9 days, about 10 days, about 12 days, about 14 days, about 16 days, about 18 days, about 20 days, about 22 days, about 24 days, about 26 days, about 28 days, about 30 days, about 1 month, about 2 months, about 3 months, about 4 months, about 5 months, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months about 22 months, about 23 months, or about 2 years. The duration of time between collection of two biological samples can be at least about 1 minute, at least about 5 minutes, at least about 10 minutes, at least about 15 minutes, at least about 30 minutes, at least about 45 minutes, at least about 1 hour, at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 24 days, at least about 28 days, at least about 1 month, at least about 2 months, at least about 4 months, at least about 6 months, at least about 8 months, at least about 10 months, at least about 1 year, at least about 1.5 years, or at least about 2 years. In some embodiments, at least two of the biological samples are collected from a subject at different occasions that are temporally separated by at least about 1 day.

Methods described herein can be repeated over the duration of a treatment or following a treatment (e.g., following a chemotherapy) of a patient. The number of repetitions of a method over the course of treatment can be about 1, about 2, about 3, about 4, about 5, about 6, about 7, about 8, about 9, about 10, about 11, about 12, about 13, about 14, or about 15.

The duration of time between repetitions of the method can be about 1 day, about 2 days, about 3 days, about 4 days, about 5 days, about 6 days, about 7 days, about 8 days, about 9 days, about 10 days, about 12 days, about 14 days, about 16 days, about 18 days, about 20 days, about 22 days, about 24 days, about 26 days, about 28 days, about 30 days, about 1 month, about 2 months, about 3 months, about 4 months, about 5 months, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months about 22 months, about 23 months, or about 2 years. The duration of time between repetitions of the method can be at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 24 days, at least about 28 days, at least about 1 month, at least about 2 months, at least about 4 months, at least about 6 months, at least about 8 months, at least about 10 months, at least about 1 year, at least about 1.5 years, or at least about 2 years.

### Sequencing

Methods described herein can include the steps of nucleic acid sequencing of biological samples and subsequent sequencing of biological samples (e.g., representative biological samples). Any nucleic acid sequencing technique (e.g., any sequencing technique for nucleic acid sequencing, any sequencing technique for subsequent sequencing) can be used in methods described herein. Suitable sequencing techniques include, but are not limited to whole exome sequencing, whole genome sequencing, RNA sequencing, single cell sequencing (e.g., single cell RNA sequencing, single cell DNA sequencing, single cell whole exome sequencing, single cell whole genome sequencing), targeted panel sequencing (e.g., gene panel sequencing), real-time PCR, deep sequencing, high-throughput sequencing (e.g., next generation sequencing), or combinations thereof. Suitable sequencing techniques can also include, but are not limited to pyrosequencing, sequencing-by synthesis, single-molecule sequencing, nanopore sequencing, semiconductor sequencing, sequencing-by-synthesis, sequencing-by-ligation, sequencing-by-hybridization, RNA-Sew (Illumina), Digital Gene Expression (Helicos), next generation sequencing, Single Molecule Sequencing by Synthesis (SMSS) (Helicos), massively-parallel sequencing, Clonal Single Molecule Array (Solexa), shotgun sequencing, Maxam-Hilbery or Sanger sequencing, primer walking, sequencing using PacBio, SOLid, Ion Torrent platform, Nanopore platform, or combinations thereof. In some embodiments, the nucleic acid sequencing is by a sequencing technique that is whole exome sequencing, whole genome sequencing, RNA sequencing, single cell sequencing, targeted panel sequencing, or combinations thereof. In some embodiments, the subsequent sequencing is by a sequencing technique that is whole exome sequencing, whole genome sequencing, RNA sequencing, single cell sequencing, targeted panel sequencing, or combinations thereof.

Methods described herein can use at least two different sequencing techniques at any step of the method, such as for the steps of nucleic acid sequencing or subsequent sequencing of a biological sample. The number of different sequencing techniques used for a step of the method can be one, two, three, four, five, six, seven, eight, nine, 10, 11, 12, 13, 14, 15, 16, 17, 18, 19, or 20. For example, the step of nucleic acid sequencing can be by whole genome sequencing and RNA sequencing of a biological sample. As another example, the step of subsequent sequencing of a biological sample can be by whole genome sequencing. As another example, the step of nucleic acid sequencing can be by whole genome sequencing, RNA sequencing, and whole exome sequencing. The number of different sequencing techniques used for a step of the method can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20. The number of different sequencing techniques used for a step of the method can be between about 1 to about 20, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 11 to about 20, about 12 to about 20, about 13 to about 20, about 14 to about 20, about 15 to about 20, about 16 to about 20, about 17 to about 20, about 18 to about 20, about 19 to about 20, about 2 to about 3, about 2 to about 4, about 2 to about 5, about 3 to about 4, about 3 to about 5, about 4 to about 5, or about 4 to about 6. In some embodiments, at least two different sequencing techniques are used in the step of nucleic acid sequencing of biological samples (e.g., two or more biological samples). In some embodiments, at least two different sequencing techniques are used in the step of subsequent sequencing of a biological sample (e.g., one or more biological samples).

Methods described herein can use the same sequencing technique for each biological sample (e.g., each of the two or more biological samples, representative biological samples) in any step (e.g., the step of nucleic acid sequencing of a biological sample, the step of subsequent sequencing of a biological sample). Methods described herein can use different sequencing techniques for biological samples (e.g., the sequencing techniques are different between two or more biological samples). Sequencing techniques can be different for any number of biological samples, such as at least about two, at least about three, at least about four, at least about five, at least about six, at least about seven, at least about eight, at least about nine, at least about ten, at least about 11, at least about 12, at least about 13, at least about 14, or at least 15 biological samples. Sequencing techniques can be different for 2, 3, 4, 5, 6, 7, 8, 9, 10, 11, 12, 13, 14, or 15 biological samples. Sequencing techniques can be different for a number of biological samples between about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2. In some embodiments, at least two biological samples (e.g., of the two or more biological samples) are sequenced by different sequencing techniques. In some embodiments, at least two biological samples (e.g., of the two or more biological samples) are sequenced by different sequencing techniques in the step of nucleic acid sequencing of a biological sample. In some embodiments, at least two biological samples (e.g., of the two or more biological samples, representative biological samples) are sequenced by different sequencing techniques in the step of subsequent sequencing of a biological sample.

Any biological sample can be submitted to the step of subsequent sequencing in methods described herein. For example, a representative sample can be submitted to subsequent sequencing. As another example, two or more biological samples combined to form a union sequencing result can be submitted to subsequent sequencing. The decision to submit as sample for subsequent sequencing can be based on sequencing parameters of sequencing results (e.g., initial sequencing results, union sequencing results, representative sequencing results of a representative biological sample, or combinations thereof). The sequencing technique used for subsequent sequencing can be different than the sequencing technique used for initial nucleic acid sequencing. For example, a method could contain the step of initial nucleic acid sequencing by whole-exome sequencing and the step of subsequent sequencing by whole genome sequencing. Subsequent sequencing of a biological sample yields a subsequent sequencing result. Methods described herein can include the step of, based on an analysis (e.g., a comparison) of the sequencing parameters (e.g., sequencing parameters of subsequent sequencing results), combining subsequent sequencing results of two or more biological samples to yield union sequencing results. The step of selecting a representative biological sample can be based on the sequencing parameters of subsequent sequencing results. Scoring of predicted immunogenicity of neoantigens in biological samples (e.g., a representative biological sample, biological samples of that are combined to form a union sequencing result) can be based on subsequent sequencing of biological samples. In some embodiments, methods contain the step of subsequent sequencing of a biological sample or a representative biological sample.

### Nucleic Acids

Sequencing (e.g., initial sequencing, subsequent sequencing) in methods described herein can be of any type of nucleic acid. Suitable nucleic acids include but are not limited to ribonucleic acids (RNA), deoxyribonucleic acids (DNA), or combinations thereof. The nucleic acids (e.g., RNA, DNA) can include the ribonucleic acid nucleotides (e.g., adenosine, uridine, cytidine, and guanosine) and/or the deoxyribonucleic acid nucleotides (e.g., 2′-deoxyadenosine, thymidine, 2′-deoxycytidine, and 2′-deoxyguanosine). The DNA and RNA can include any modified (e.g., post-translationally modified, chemically modified) nucleotides including, but not limited to, 1-methyladenosine, 6-methyladenosine, 2′-O-methyl-adenosine, 5-methyl-cytosine, 6-methyl-guanosine, 4-acetyl-cytidine, pseudouridine, N1-methylpseudouridine, 5-hydroxymethyl-cytidine, inosine, or combinations thereof.

Methods described herein can sequence any type of DNA. Suitable types of DNA for sequencing include, but are not limited to, genomic DNA, complementary DNA (cDNA), plasmid DNA, mitochondrial DNA, or combinations thereof. Methods described herein can sequence any type of RNA. Suitable types of RNA for sequencing include, but are not limited to, transfer RNA (tRNA), small nuclear RNA (snRNA), Piwi-interacting RNA, ribosomal RNA (rRNA), messenger RNA (mRNA), non-coding RNA (ncRNA), small interfering RNA (siRNA), small nucleolar RNA (snoRNA), precursor mRNA (pre-mRNA), or combinations thereof.

The steps of nucleic acid sequencing and/or subsequent sequencing of biological samples can include sequencing of a nucleic acid that is RNA, DNA, or a combination thereof. In some embodiments, the step of nucleic acid sequencing is sequencing of a nucleic acid that is RNA, DNA, or a combination thereof. In some embodiments, the step of subsequent sequencing (e.g., subsequent nucleic acid sequencing) is sequencing of a nucleic acid that is RNA, DNA, or a combination thereof.

### Analysis of Sequencing Parameters

Sequencing results obtained by methods described herein (e.g., initial nucleic acid sequencing results, subsequent sequencing results) can be any type of nucleic acid sequencing results including, but not limited to, raw nucleic acid sequencing reads (e.g., raw DNA sequence reads, raw RNA sequence reads, raw exome sequence reads, raw whole genome sequence reads), sequencing variants (e.g., DNA sequencing variants compared to healthy tissue of a subject, RNA sequencing variants compared to healthy tissue of a subject, protein modifying sequencing variants), predicted RNA sequences based on transcription of DNA sequence reads, predicted neoantigen peptides (e.g., neoantigen peptides based on RNA sequence reads, neoantigen peptides based on DNA sequence reads), or combinations thereof. Sequencing variants can be somatic variants. Sequencing variants can be germline variants. Sequencing variants can be a combination of somatic and germline variants. Sequencing results can be sequencing reads, sequencing variants, encoded peptides, or combinations thereof. In some embodiments, the initial sequencing results (e.g., the results of nucleic acid sequencing of a biological sample, the results of subsequent sequencing of a biological sample) can be sequencing reads, sequencing variants, encoded peptides, or a combination thereof. In some embodiments, the subsequent sequencing results (e.g., the results of the step of subsequent sequencing of a biological sample, the results of the step of nucleic acid sequencing of a biological sample) can be sequencing reads, sequencing variants, encoded peptides, or a combination thereof.

Sequencing results of methods described herein (e.g., union sequencing results, initial sequencing results, subsequent sequencing results) can have one or more sequencing parameters based on the sequencing technique used and the type of sequencing results obtained. Sequencing parameters can be any parameter of a sequencing result (e.g., initial nucleic acid sequencing result, subsequent sequencing result) including, but not limited to, number of sequence reads (e.g., number of total sequence reads), number of sequencing variants (e.g., number of DNA sequencing variants, number of RNA sequencing variants), tumor purity, sequencing depth, number of protein modifying sequencing variants (e.g., number of protein-modifying sequencing variants confirmed by RNA), RNA confirmation rate, RNA quality, number of long identified neoantigen peptides, number of short identified neoantigen peptides, predicted immunogenicity of a neoantigen peptide, or combinations thereof. The number of sequence reads parameter can be the number of DNA sequence reads, RNA sequence reads, or a combination thereof. The number of sequencing variants (e.g., DNA sequencing variants, RNA sequencing variants, encoded peptide variants, or a combination thereof) can be the variants identified in comparison of two samples from a subject (e.g., a tumor sample and a non-tumor sample) or in comparison of a sample of a subject with a reference sample (e.g., a tumor sample of a subject compared with a reference human genome). Tumor purity is the percentage or ratio of tumor sequence reads to healthy cell sequence reads (e.g., healthy tissue sequence reads). A higher tumor purity indicates less non-tumor genetic material and/or sequence reads present in a biological sample. Sequencing depth represents the number of nucleotides contributing to a portion of an assembly. For example, a sequencing depth of 10× indicates each nucleobase has been sequenced an average of 10 times in 10 different read sequences. The number of protein-modifying sequencing variants can be the number of RNA variants, DNA variants, or a combination thereof that are predicted to encode for a protein variant. Protein variants can be determined by any method. For example, the number of protein variants can be determined by comparison of healthy tissue proteins of a subject to tumor tissue proteins of a subject. As another example, the number of protein variants can be determined by comparison of reference sequence (e.g., a reference exome, a reference genome, a reference proteome) proteins or encoded proteins to the proteins encoded in a sequencing results of a biological sample from a subject (e.g., proteins encoded by a DNA sequencing result, an RNA sequencing result, or a combination thereof of a biological sample). RNA confirmation rate is the rate (e.g., ratio, percentage) of protein-modifying variants that are identified by nucleic acid sequencing and confirmed by RNA sequencing (e.g., number of protein-modifying variants confirmed by RNA sequencing (e.g., bulk RNA sequencing) divided by total sequencing variants). For example, if 80 protein-modifying variants are observed by DNA sequencing and 25 protein-modifying variants are confirmed by RNA sequencing, the RNA confirmation rate is 0.3125 (i.e., 31.25%). RNA quality can be assessed by any methodology, including but not limited to RNA integrity number (RIN, e.g., see Schroeder, A. et al. The RIN: an RNA integrity number for assigning integrity values to RNA measurements. (2006) BMC Bioinformatics 7, 3, 1-14), transcript integrity number (TIN, e.g., see Wang, L. et al. Measure transcript integrity using RNA-seq data. (2016) BMC Bioinformatics 17, 58, 1-16), or a combination thereof. The number of identified neoantigen peptides (e.g., long identified neoantigen peptides, short identified neoantigen peptides, a combination of long and short identified neoantigen peptides), such as the number of long identified neoantigen peptides and/or the number of short identified neoantigen peptides, can be a sequence parameter for analysis (e.g., comparison) of biological sample sequencing results. The predicted immunogenicity of any number of neoantigen peptides can be a sequencing parameter. The number of neoantigen peptides with predicted immunogenicity can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. The number of neoantigen peptides with predicted immunogenicity can be between about 1 to about 3500, about 1 to about 3200, about 1 to about 2800, about 1 to about 2600, about 1 to about 2400, about 1 to about 2200, about 1 to about 2000, about 1 to about 1900, about 1 to about 1800, about 1 to about 1700, about 1 to about 1600, about 1 to about 1500, about 1 to about 1400, about 1 to about 1300, about 1 to about 1200, about 1 to about 1000, about 1 to about 1000, about 1 to about 950, about 1 to about 900, about 1 to about 850, about 1 to about 800, about 1 to about 750, about 1 to about 700, about 1 to about 650, about 1 to about 600, about 1 to about 550, about 1 to about 500, about 1 to about 480, about 1 to about 460, about 1 to about 440, about 1 to about 420, about 1 to about 400, about 1 to about 380, about 1 to about 360, about 1 to about 340, about 1 to about 320, about 1 to about 300, about 1 to about 280, about 1 to about 260, about 1 to about 240, about 1 to about 220, about 1 to about 200, about 1 to about 190, about 1 to about 180, about 1 to about 170, about 1 to about 160, about 1 to about 150, about 1 to about 140, about 1 to about 130, about 1 to about 120, about 1 to about 110, about 1 to about 100, about 1 to about 90, about 1 to about 85, about 1 to about 80, about 1 to about 75, about 1 to about 70, about 1 to about 65, about 1 to about 60, about 1 to about 55, about 1 to about 50, about 1 to about 45, about 1 to about 40, about 1 to about 35, about 1 to about 30, about 1 to about 29, about 1 to about 28, about 1 to about 27, about 1 to about 26, about 1 to about 25, about 1 to about 24, about 1 to about 23, about 1 to about 22, about 1 to about 21, about 1 to about 20, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 100, about 3 to about 100, about 4 to about 100, about 5 to about 100, about 6 to about 100, about 7 to about 100, about 8 to about 100, about 9 to about 100, about 10 to about 100, about 11 to about 100, about 12 to about 100, about 13 to about 100, about 14 to about 100, about 15 to about 100, about 16 to about 100, about 17 to about 100, about 18 to about 100, about 19 to about 100, about 20 to about 100, about 22 to about 100, about 24 to about 100, about 26 to about 100, about 28 to about 100, about 30 to about 100, about 33 to about 100, about 36 to about 100, about 40 to about 100, about 44 to about 100, about 48 to about 100, about 50 to about 100, about 55 to about 100, about 60 to about 100, about 65 to about 100, about 70 to about 100, about 75 to about 100, about 80 to about 100, about 85 to about 100, about 90 to about 100, about 95 to about 100, or about 2 to about 100. The number of neoantigen peptides with predicted immunogenicity can be at least about 1, at least about 2, at least about 3, at least about 4, at least about 5, at least about 6, at least about 7, at least about 8, at least about 9, at least about 10, at least about 12, at least about 14, at least about 16, at least about 18, at least about 20, at least about 24, at least about 28, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 120, at least about 140, at least about 160, at least about 180, at least about 200, at least about 240, at least about 280, at least about 300, at least about 350, at least about 400, at least about 450, or at least about 500. In some embodiments, one or more sequencing parameters are a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, or a combination thereof.

As used herein, the terms “short identified neoantigen peptide” and “short peptide” can refer to a peptide with an amino acid length that is between about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, about 3 to about 4, about 4 to about 15, about 5 to about 15, about 6 to about 15, about 7 to about 15, about 8 to about 15, about 9 to about 15, about 10 to about 15, about 11 to about 15, about 12 to about 15, about 13 to about 15, about 14 to about 15, about 8 to about 11, about 8 to about 10, about 8 to about 9, about 9 to about 11, about 10 to about 11, about 7 to about 11, about 6 to about 11, about 5 to about 11, about 4 to about 11, about 3 to about 11, about 8 to about 12, about 8 to about 13, or about 8 to about 14. “Short identified neoantigen peptide” and “short peptide” can refer to a peptide with an amino acid length that is about 3, about 4, about 5, about 6, about 7, about 8, about 9, about 10, about 11, about 12, about 13, about 14, or about 15. Short identified neoantigen peptide” and “short peptide” can refer to a peptide with an amino acid length that is at most about 15, at most about 14, at most about 13, at most about 12, at most about 11, at most about 10, at most about 9, at most about 8, at most about 7, at most about 6, at most about 5, at most about 4, at most about 3, or at most about 2. As used herein, the terms “long identified neoantigen peptide” and “long peptide” can refer to a peptide with an amino acid length that is between about 13 and about 30, about 13 and about 29, about 13 and about 28, about 13 and about 27, about 13 and about 26, about 13 and about 25, about 13 and about 24, about 13 and about 23, about 13 and about 22, about 13 and about 21, about 13 and about 20, about 13 and about 19, about 13 and about 18, about 13 and about 17, about 13 and about 16, about 13 and about 15, about 13 and about 14, about 14 and about 30, about 15 and about 30, about 16 and about 30, about 17 and about 30, about 18 and about 30, about 19 and about 30, about 20 and about 21, about 22 and about 30, about 23 and about 30, about 24 and about 30, about 25 and about 30, about 26 and about 30, about 27 and about 30, about 28 and about 30, about 29 and about 30, or about 13 and about 25. “Long identified neoantigen peptide” and “long peptide” can refer to a peptide with an amino acid length that is about 13, about 14, about 15, about 16, about 17, about 18, about 19, about 20, about 21, about 22, about 23, about 24, about 25, about 26, about 27, about 28, about 29, or about 30. “Long identified neoantigen peptide” and “long peptide” can refer to a peptide with an amino acid length that is at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30.

Any number of sequencing parameters of sequencing results (e.g., of initial sequencing results) can be analyzed (e.g., compared between biological samples (e.g., between two or more biological samples)). Without wishing to be bound by any one particular theory, it is believed that an analysis of sequencing parameters (e.g., a comparison sequencing parameters between biological samples) can be used in determining to combine initial sequencing results of two or more biological samples to yield union sequencing results or to select a representative biological sample. It is further believed that this methodology affords the most representative result of genomic variance in the originating tissue of the biological samples (e.g., a combination of samples of similar quality, or selection of the best quality sample) and that this representative result can be used to generate a treatment for a subject (e.g., generate a neoantigen vaccine). Any sequencing parameters can be analyzed (e.g., compared between biological samples) including, but not limited to number of sequence reads (e.g., number of total sequence reads), number of sequencing variants (e.g., number of DNA sequencing variants, number of RNA sequencing variants), tumor purity, sequencing depth, number of protein-modifying sequencing variants (e.g., number of protein-modifying sequencing variants confirmed by RNA), RNA confirmation rate, RNA quality, number of long identified neoantigen peptides, number of short identified neoantigen peptides, predicted immunogenicity of a neoantigen peptide, or combinations thereof. The number of different sequencing parameters analyzed (e.g., compared between biological samples) can be about 1, about 2, about 3, about 4, about 5, about 6, about 7, about 8, about 9, about 10, about 11, about 12, about 13, about 14, or about 15. The number of different sequencing parameters analyzed (e.g., compared between biological samples) can be between about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 15, about 3 to about 15, about 4 to about 15, about 5 to about 15, about 6 to about 15, about 7 to about 15, about 8 to about 15, about 2 to about 3, about 2 to about 4, about 2 to about 5, about 2 to about 6, about 2 to about 7, about 2 to about 8, about 2 to about 9, about 2 to about 10, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, or about 4 to about 10. The number of different sequencing parameters analyzed (e.g., compared between biological samples) can be at least about 1, at least about 2, at least about 3, at least about 4, at least about 5, at least about 6, at least about 7, at least about 8, at least about 9, or at least about 10. For example, the sequencing results of a first biological sample and a second biological sample can be compared based on the number of long identified neoantigen peptides in the first biological sample and the second biological sample. Continuing this example, if the numbers of long identified neoantigen peptides are similar (e.g., less than 20% difference, a ratio of between the sequencing parameters of less than 1.20) between both the first and second biological sample, then the initial sequencing results of both first and second samples are combined to yield union sequencing results. As another example, the sequencing results of a first biological sample and a second biological sample can be analyzed (e.g., compared) based on the tumor purity, the number of protein-modifying variants, and the RNA quality of the first biological sample and the second biological sample. Continuing this example, the sample with the highest tumor purity, number of protein-modifying variants, and RNA quality can be selected as a representative biological sample.

Sequencing parameters of any similarity between biological samples can indicate that combination of the sequencing results of the biological samples for union sequencing results is appropriate. Sequencing parameters can be analyzed (e.g., compared) by any mathematical calculation. For example, a ratio between sequencing parameter values of sequencing results can be generated. In such a ratio, the larger of the two sequencing parameter values can be the numerator (e.g., A:B or A/B, wherein the sequencing parameter A is larger in value or equal to the sequencing parameter B). A ratio (e.g., a ratio that indicates sequencing results will be combined to form a union of sequencing results) of a first biological sample sequencing parameter value and a second biological sample sequencing parameter value can be at most about 1.05, at most about 1.10, at most about 1.15, at most about 1.20, at most about 1.25, at most about 1.30, at most about 1.35, at most about 1.40, at most about 1.45, at most about 1.50, at most about 1.55, at most about 1.60, at most about 1.65, at most about 1.70, at most about 1.75, at most about 1.80, at most about 1.85, at most about 1.90, at most about 1.95, at most about 2.00, at most about 2.05, at most about 2.10, at most about 2.15, at most about 2.20, at most about 2.25, at most about 2.30, at most about 2.35, at most about 2.40, at most about 2.45, at most about 2.50, at most about 2.55, at most about 2.60, at most about 2.65, at most about 2.70, at most about 2.75, at most about 2.80, at most about 2.85, at most about 2.90, at most about 2.95, at most about 3.00, at most about 3.10, at most about 3.20, at most about 3.30, at most about 3.40, at most about 3.50, at most about 3.60, at most about 3.70, at most about 3.80, at most about 3.90, or at most about 4.00. A ratio (e.g., a ratio that indicates sequencing results will be combined to form a union of sequencing results) of a first biological sample sequencing parameter value and a second biological sample sequencing parameter value can be at about 1.00, about 1.05, about 1.10, about 1.15, about 1.20, about 1.25, about 1.30, about 1.35, about 1.40, about 1.45, about 1.50, about 1.55, about 1.60, about 1.65, about 1.70, about 1.75, about 1.80, about 1.85, about 1.90, about 1.95, about 2.00, about 2.05, about 2.10, about 2.15, about 2.20, about 2.25, about 2.30, about 2.35, about 2.40, about 2.45, about 2.50, about 2.55, about 2.60, about 2.65, about 2.70, about 2.75, about 2.80, about 2.85, about 2.90, about 2.95, about 3.00, about 3.10, about 3.20, about 3.30, about 3.40, about 3.50, about 3.60, about 3.70, about 3.80, about 3.90, or about 4.00. A ratio (e.g., a ratio that indicates sequencing results will be combined to form a union of sequencing results) of a first biological sample sequencing parameter value and a second biological sample sequencing parameter value can be between about 1.00 to about 4.00, about 1.00 to about 3.80, about 1.00 to about 3.60, about 1.00 to about 3.40, about 1.00 to about 3.20, about 1.00 to about 3.00, about 1.00 to about 2.80, about 1.00 to about 2.60, about 1.00 to about 2.40, about 1.00 to about 2.20, about 1.00 to about 2.00, about 1.00 to about 1.90, about 1.00 to about 1.80, about 1.00 to about 1.70, about 1.00 to about 1.60, about 1.00 to about 1.50, about 1.00 to about 1.40, about 1.00 to about 1.30, about 1.00 to about 1.20, about 1.00 to about 1.10, or about 1.00 to about 1.05. For example, the number of long identified neoantigen peptides could be 150 for a first biological sample and 100 for a second biological sample (i.e., a ratio of 1.50, a ratio of at most about 1.55, a ratio of about 1.00 to about 1.60), indicating the sampling results should be combined to yield a union sequencing result.

Sequencing parameters of any similarity between biological samples can indicate that combination of the sequencing results of the biological samples for union sequencing results is not appropriate and/or that a biological sample should be selected for as a representative biological sample in methods described herein. Sequencing parameters can be analyzed (e.g., compared) by any mathematical calculation, such as a ratio. In such a ratio, the larger of the two sequencing parameter values can be the numerator (e.g., A:B or A/B, wherein the sequencing parameter A is larger in value or equal to the sequencing parameter B). A ratio (e.g., a ratio that indicates sequencing results will not be combined to form a union of sequencing results, a ratio that indicates a biological sample will be selected as a representative biological sample) of a first biological sample sequencing parameter value and a second biological sample sequencing parameter value can at least about 1.50, at least about 1.55, at least about 1.60, at least about 1.65, at least about 1.70, at least about 1.75, at least about 1.80, at least about 1.85, at least about 1.90, at least about 1.95, at least about 2.00, at least about 2.05, at least about 2.10, at least about 2.15, at least about 2.20, at least about 2.25, at least about 2.30, at least about 2.35, at least about 2.40, at least about 2.45, at least about 2.50, at least about 2.55, at least about 2.60, at least about 2.65, at least about 2.70, at least about 2.75, at least about 2.80, at least about 2.85, at least about 2.90, at least about 2.95, at least about 3.00, at least about 3.10, at least about 3.20, at least about 3.30, at least about 3.40, at least about 3.50, at least about 3.60, at least about 3.70, at least about 3.80, at least about 3.90, at least about 4.00, at least about 4.50, at least about 5.00, at least about 5.50, at least about 6.00, at least about 6.50, at least about 7.50, at least about 8.00, at least about 8.50, at least about 9.00, at least about 9.50, or at least about 10.00. A ratio (e.g., a ratio that indicates a biological sample will be selected as a representative biological sample) of a first biological sample sequencing parameter value and a second biological sample sequencing parameter value can be about 1.50, about 1.55, about 1.60, about 1.65, about 1.70, about 1.75, about 1.80, about 1.85, about 1.90, about 1.95, about 2.00, about 2.05, about 2.10, about 2.15, about 2.20, about 2.25, about 2.30, about 2.35, about 2.40, about 2.45, about 2.50, about 2.55, about 2.60, about 2.65, about 2.70, about 2.75, about 2.80, about 2.85, about 2.90, about 2.95, about 3.00, about 3.10, about 3.20, about 3.30, about 3.40, about 3.50, about 3.60, about 3.70, about 3.80, about 3.90, about 4.00, about 4.50, about 5.00, about 5.50, about 6.00, about 6.50, about 7.00, about 7.50, about 8.00, about 8.50, about 9.00, about 9.50, or about 10.00. A ratio (e.g., a ratio that indicates a biological sample will be selected as a representative biological sample) of a first biological sample sequencing parameter value and a second biological sample sequencing parameter value can be between about 1.50 to about 10.00, about 1.75 to about 10.00, about 2.00 to about 10.00, about 2.25 to about 10.00, about 2.50 to about 10.00, about 3.00 to about 10.00, about 3.50 to about 10.00, about 4.00 to about 10.00, about 4.50 to about 10.00, about 5.00 to about 10.00, about 5.50 to about 10.00, about 6.00 to about 10.00, about 6.50 to about 10.00, about 7.00 to about 10.00, about 7.50 to about 10.00, about 8.00 to about 10.00, about 8.50 to about 10.00, about 9.00 to about 10.00, about 9.50 to about 10.00, about 1.50 to about 9.50, about 1.50 to about 9.0, about 1.50 to about 8.50, about 1.50 to about 8.00, about 1.50 to about 7.50, about 1.50 to about 7.00, about 1.50 to about 6.50, about 1.50 to about 6.00, about 1.50 to about 5.50, about 1.50 to about 5.00, about 1.50 to about 4.50, about 1.50 to about 4.00, about 1.50 to about 3.80, about 1.50 to about 3.60, about 1.50 to about 3.40, about 1.50 to about 3.20, about 1.50 to about 3.00, about 1.50 to about 2.80, about 1.50 to about 2.60, about 1.50 to about 2.40, about 1.50 to about 2.20, about 1.50 to about 2.00, about 1.50 to about 1.90, about 1.50 to about 1.80, about 1.50 to about 1.70, or about 1.50 to about 1.60. For example, the number of long identified neoantigen peptides could be 1000 for a first biological sample and 200 for a second biological sample (i.e., a ratio of 5.00), indicating that the first biological sample will be selected as a representative biological sample.

Sequencing parameters can be used to evaluate the quality of a sequencing result (e.g., a sequencing result of a biological sample from a patient). A value of zero for any sequencing parameter of a sequencing result described herein can indicate the corresponding biological sample should not be selected as a representative biological sample or should not be combined with another biological sample to yield a union sequencing result. The value of a sequencing parameter can indicate the appropriateness of a sample for selection by methods described herein, indicate the biological sample is not appropriate for selection or combination with the sequencing result of different biological sample, or indicate another biological sample must be collected from a patient. A number of sequencing reads (e.g., number of total sequence reads) of a sequencing result that is zero can indicate the sample is not appropriate for methods or method steps described herein (e.g., not appropriate for selection or combination with the sequencing results of a different sample, indicate another biological sample must be collected from a patient). A number of sequencing variants (e.g., number of DNA sequencing variants, number of RNA sequencing variants) of a sequencing result that is zero can indicate the sample is not appropriate for methods or method steps described herein (e.g., not appropriate for selection of a representative biological sample or combination with the sequencing results of a different sample, indicate another biological sample must be collected from a patient). A tumor purity of a sequencing result that is zero can indicate the sample is not appropriate for methods or method steps described herein (e.g., not appropriate for selection of a representative biological sample or combination with the sequencing results of a different sample, indicate another biological sample must be collected from a patient). A sequencing depth of a sequencing result that is zero can indicate the sample is not appropriate for methods or method steps described herein (e.g., not appropriate for selection of a representative biological sample or combination with the sequencing results of a different sample, indicate another biological sample must be collected from a patient). A number of protein-modifying sequencing variants (e.g., number of protein-modifying sequencing variants confirmed by RNA) of a sequencing result that is zero can indicate the sample is not appropriate for methods or method steps described herein (e.g., not appropriate for selection of a representative biological sample or combination with the sequencing results of a different sample, indicate another biological sample must be collected from a patient). An RNA confirmation rate of a sequencing result that is zero can indicate the sample is not appropriate for methods or method steps described herein (e.g., not appropriate for selection of a representative biological sample or combination with the sequencing results of a different sample, indicate another biological sample must be collected from a patient). An RNA quality of a sequencing result that is zero can indicate the sample is not appropriate for methods or method steps described herein (e.g., not appropriate for selection of a representative biological sample or combination with the sequencing results of a different sample, indicate another biological sample must be collected from a patient). A number of long identified neoantigen peptides of a sequencing result that is zero can indicate the sample is not appropriate for methods or method steps described herein (e.g., not appropriate for selection of a representative biological sample or combination with the sequencing results of a different sample, indicate another biological sample must be collected from a patient). A number of short identified neoantigen peptides of a sequencing result that is zero can indicate the sample is not appropriate for methods or method steps described herein (e.g., not appropriate for selection of a representative biological sample or combination with the sequencing results of a different sample, indicate another biological sample must be collected from a patient). A predicted immunogenicity of a neoantigen peptide of a sequencing result that is zero can indicate the sample is not appropriate for methods or method steps described herein (e.g., not appropriate for selection of a representative biological sample or combination with the sequencing results of a different sample, indicate another biological sample must be collected from a patient).

### Combining Sequencing Results—Union Sequencing Results

Based on an analysis (e.g., a comparison) of sequencing parameters (e.g., one or more sequencing parameters) of the initial sequencing results, the sequencing results of two or more biological samples can be combined to yield union sequencing results. The sequencing results can be combined using any methodology, including but not limited to concatenating of sequencing results (e.g., sequence reads), averaging of sequence read frequency (e.g., weighted averaging of sequence read frequency), joint calling of sequence reads, taking the union of variant call sets, taking the intersection of variant call sets, or combinations thereof.

The union sequencing results can be obtained by combination of the sequencing results of any number of biological samples. The number of biological samples (e.g., the number of biological sample sequencing results) combined to generate union sequence results can be 2, 3, 4, 5, 6, 7, 8, 9, 10, 11, 12, 13, 14, 15, 16, 17, 18, 19, or 20. The number of biological samples (e.g., the number of biological sample sequencing results) combined to generate union sequence results can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20. The number of biological samples (e.g., the number of biological sample sequencing results) combined to generate union sequence results can be at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20. The number of biological samples (e.g., the number of biological sample sequencing results) combined to generate union sequence results can be between about 2 to about 20, about 2 to about 18, about 2 to about 16, about 2 to about 14, about 2 to about 12, about 2 to about 10, about 2 to about 9, about 2 to about 8, about 2 to about 7, about 2 to about 6, about 2 to about 5, about 2 to about 4, about 2 to about 3, about 3 to about 20, about 4 to about 20, about 5 to about 20, about 6 to about 20, about 7 to about 20, about 8 to about 20, about 9 to about 20, about 10 to about 20, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10.

The union sequencing results can be any type of nucleic acid sequencing results including, but not limited to, raw nucleic acid sequencing reads (e.g., raw DNA sequence reads, raw RNA sequence reads, raw exome reads, raw whole genome reads), sequencing variants (e.g., DNA sequencing variants compared to healthy cells or healthy tissue of a subject, RNA sequencing variance compared to healthy cells or healthy tissue of a subject, protein modifying sequencing variants, somatic mutation variants, germline mutation variants), predicted RNA sequences based on transcription of DNA sequence reads, predicted neoantigen peptides (e.g., neoantigen peptides based on RNA sequence reads, neoantigen peptides based on DNA sequence reads), or combinations thereof. Union sequencing results can be sequencing reads, sequencing variants, encoded peptides, or combinations thereof.

Any number of union sequencing parameters of union sequencing results (e.g., sequencing parameters of union sequencing results obtained from combining initial sequencing results of two or more biological samples) can be compared to the sequencing parameters (e.g., any sequencing parameter described herein) of initial sequencing results in methods described herein. Without wishing to be bound by any one particular theory, it is believed that a comparison of one or more union sequencing parameters with one or more sequence parameters of the initial sequencing results can be used in determining whether the scoring of predicted immunogenicity of neoantigens by methods described herein is based on a union sequencing result of two or more biological samples or a representative biological sample. Any union sequencing parameter can be compared including, but not limited to number of sequence reads (e.g., number of total sequence reads), number of sequencing variants (e.g., number of DNA sequencing variants, number of RNA sequencing variants), tumor purity, sequencing depth, number of protein-modifying sequencing variants (e.g., number of protein-modifying sequencing variants confirmed by RNA), RNA confirmation rate, RNA quality, number of long identified neoantigen peptides, number of short identified neoantigen peptides, predicted immunogenicity of a neoantigen peptide, or combinations thereof. The number of different union sequencing parameters compared can be 1, 2, 3, 4, 5, 6, 7, 8, 9, 10, 11, 12, 13, 14, or 15. The number of different union sequencing parameters compared can be between about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 15, about 3 to about 15, about 4 to about 15, about 5 to about 15, about 6 to about 15, about 7 to about 15, about 8 to about 15, about 2 to about 3, about 2 to about 4, about 2 to about 5, about 2 to about 6, about 2 to about 7, about 2 to about 8, about 2 to about 9, about 2 to about 10, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, or about 4 to about 10. The number of different union sequencing parameters compared can be at least about 1, at least about 2, at least about 3, at least about 4, at least about 5, at least about 6, at least about 7, at least about 8, at least about 9, or at least about 10. For example, the union sequencing results and the initial sequencing results of a first biological sample can be compared based on the number of long identified neoantigen peptides in the union sequencing result and the first biological sample initial sequencing result. Continuing this example, if the ratio of number of long identified neoantigen peptides between the union sequencing results and the initial sequencing result of the first biological sample is higher than 1.2 (e.g., the union sequencing result contains 20% more long identified neoantigen peptides than the initial sequencing result) then the union sequencing result is used to score the predicted immunogenicity of neoantigens by methods described herein. As another example, the union sequencing results and a first biological sample initial sequencing result can be compared based on the tumor purity, the number of protein-modifying variants, and the RNA quality of the union sequencing results and the first biological sample initial sequencing result. Continuing this example, the sequencing result with the highest tumor purity, number of protein-modifying variants, and/or RNA quality can be selected to base the scoring of predicted immunogenicity of neoantigens upon. In some embodiments, the method can further comprise the step of comparing one or more union sequencing parameters of the union sequencing results to the one or more sequencing parameters of the initial sequencing results (e.g., sequencing parameters obtained by nucleic acid sequencing of a biological sample). In some embodiments, the one or more union sequencing parameters are a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, or a combination thereof.

Union sequencing parameters of any ratio (e.g., union-initial sequencing parameter ratio, union:initial sequencing parameter ratio, union sequencing parameter/initial sequencing result parameter of a tissue sample) with a sequencing parameter of an initial nucleic acid sequencing of a biological sample can indicate that the scoring of predicted immunogenicity will not be based on the union sequencing results. A union-initial sequencing parameter ratio (e.g., a ratio of union sequencing parameter value to a sequencing parameter value of an initial nucleic acid sequencing of a biological sample, a ratio that indicates the scoring of predicted immunogenicity will not be based on the union sequencing results, a ratio that indicates the scoring of predicted immunogenicity will be based on the sequencing results of a representative biological sample) can be at most about 1.00, at most about 0.95, at most about 0.90, at most about 0.85, at most about 0.80, at most about 0.75, at most about 0.70, at most about 0.65, at most about 0.60, at most about 0.55, at most about 0.50, at most about 0.45, at most about 0.40, at most about 0.35, at most about 0.30, at most about 0.25, at most about 0.20, at most about 0.15, at most about 0.10, at most about 0.05, or at most about 0.01. A union-initial sequencing parameter ratio (e.g., a ratio of union sequencing parameter value to a sequencing parameter value of an initial nucleic acid sequencing of a biological sample, a ratio that indicates the scoring of predicted immunogenicity will not be based on the union sequencing results, a ratio that indicates the scoring of predicted immunogenicity will be based on the sequencing results of a representative biological sample) can be about 0.95, about 0.90, about 0.85, about 0.80, about 0.75, about 0.70, about 0.65, about 0.60, about 0.55, about 0.50, about 0.45, about 0.40, about 0.35, about 0.30, about 0.25, about 0.20, about 0.15, about 0.10, about 0.05, or about 0.01. A union-initial sequencing parameter ratio (e.g., a ratio of union sequencing parameter value to a sequencing parameter value of an initial nucleic acid sequencing of a biological sample, a ratio that indicates the scoring of predicted immunogenicity will not be based on the union sequencing results, a ratio that indicates the scoring of predicted immunogenicity will be based on the sequencing results of a representative biological sample) can be between about 0.05 to about 0.95, about 0.10 to about 0.95, about 0.15 to about 0.95, about 0.20 to about 0.95, about 0.25 to about 0.95, about 0.30 to about 0.95, about 0.35 to about 0.95, about 0.40 to about 0.95, about 0.45 to about 0.95, about 0.50 to about 0.95, about 0.55 to about 0.95, about 0.60 to about 0.95, about 0.65 to about 0.95, about 0.70 to about 0.95, about 0.75 to about 0.95, about 0.80 to about 0.95, about 0.85 to about 0.95, about 0.90 to about 0.95, about 0.05 to about 0.90, about 0.05 to about 0.85, about 0.05 to about 0.80, about 0.05 to about 0.75, about 0.05 to about 0.70, about 0.05 to about 0.65, about 0.05 to about 0.60, about 0.05 to about 0.55, about 0.05 to about 0.50, about 0.05 to about 0.45, about 0.05 to about 0.40, about 0.05 to about 0.35, about 0.05 to about 0.30, about 0.05 to about 0.25, about 0.05 to about 0.20, about 0.05 to about 0.15, about 0.05 to about 0.12, or about 0.05 to about 0.10. For example, the number of long identified neoantigen peptides could be 100 for union sequencing results and 150 for an initial sequencing result of a biological sample (i.e., a ratio of about 0.66), indicating the scoring of predicted immunogenicity of one or more neoantigens will be based on a sequencing results of a representative biological sample and not based on the union sequencing results.

Union sequencing parameters of any ratio (e.g., union-initial sequencing parameter ratio) with a sequencing parameter of an initial nucleic acid sequencing of a biological sample can indicate that the scoring of predicted immunogenicity will be based on the union sequencing results. A union-initial sequencing parameter ratio (e.g., a ratio of union sequencing parameter value to a sequencing parameter value of an initial nucleic acid sequencing of a biological sample, a ratio that indicates the scoring of predicted immunogenicity will be based on the union sequencing results) can be at least about 1.05, at least about 1.10, at least about 1.15, at least about 1.20, at least about 1.25, at least about 1.30, at least about 1.35, at least about 1.40, at least about 1.45, at least about 1.50, at least about 1.55, at least about 1.60, at least about 1.65, at least about 1.70, at least about 1.75, at least about 1.80, at least about 1.85, at least about 1.90, at least about 1.95, at least about 2.00, at least about 2.05, at least about 2.10, at least about 2.15, at least about 2.20, at least about 2.25, at least about 2.30, at least about 2.35, at least about 2.40, at least about 2.45, at least about 2.50, at least about 2.55, at least about 2.60, at least about 2.65, at least about 2.70, at least about 2.75, at least about 2.80, at least about 2.85, at least about 2.90, at least about 2.95, at least about 3.00, at least about 3.10, at least about 3.20, at least about 3.30, at least about 3.40, at least about 3.50, at least about 3.60, at least about 3.70, at least about 3.80, at least about 3.90, at least about 4.00, at least about 4.50, at least about 5.00, at least about 5.50, at least about 6.00, at least about 6.50, at least about 7.00, at least about 7.50, at least about 8.00, at least about 8.50, at least about 9.00, at least about 9.50, or at least about 10.00. A union-initial sequencing parameter ratio (e.g., a ratio of union sequencing parameter value to a sequencing parameter value of an initial nucleic acid sequencing of a biological sample, a ratio that indicates the scoring of predicted immunogenicity will be based on the union sequencing results,) can be about 1.05, about 1.10, about 1.15, about 1.20, about 1.25, about 1.35, about 1.40, about 1.45, about 1.50, about 1.55, about 1.60, about 1.65, about 1.70, about 1.75, about 1.80, about 1.85, about 1.90, about 1.95, about 2.00, about 2.05, about 2.10, about 2.15, about 2.20, about 2.25, about 2.30, about 2.35, about 2.40, about 2.45, about 2.50, about 2.55, about 2.60, about 2.65, about 2.70, about 2.75, about 2.80, about 2.85, about 2.90, about 2.95, about 3.00, about 3.10, about 3.20, about 3.30, about 3.40, about 3.50, about 3.60, about 3.70, about 3.80, about 3.90, about 4.00, about 4.50, about 5.00, about 5.50, about 6.00, about 6.50, about 7.00, about 7.50, about 8.00, about 8.50, about 9.00, about 9.50, or about 10.00. A union-initial sequencing parameter ratio (e.g., a ratio of union sequencing parameter value to a sequencing parameter value of an initial nucleic acid sequencing of a biological sample, a ratio that indicates the scoring of predicted immunogenicity will be based on the union sequencing results,) can be between about 1.05 to about 10.00, about 1.10 to about 10.00, about 1.15 to about 10.00, about 1.20 to about 10.00, about 1.25 to about 10.00, about 1.30 to about 10.00, about 1.35 to about 10.00, about 1.40 to about 10.00, about 1.45 to about 10.00, about 1.50 to about 10.00, about 1.75 to about 10.00, about 2.00 to about 10.00, about 2.25 to about 10.00, about 2.50 to about 10.00, about 3.00 to about 10.00, about 3.50 to about 10.00, about 4.00 to about 10.00, about 4.50 to about 10.00, about 5.00 to about 10.00, about 5.50 to about 10.00, about 6.00 to about 10.00, about 6.50 to about 10.00, about 7.00 to about 10.00, about 7.50 to about 10.00, about 8.00 to about 10.00, about 8.50 to about 10.00, about 9.00 to about 10.00, about 9.50 to about 10.00, about 1.05 to about 9.50, about 1.05 to about 9.00, about 1.05 to about 8.50, about 1.05 to about 8.00, about 1.05 to about 7.50, about 1.05 to about 7.00, about 1.05 to about 6.50, about 1.05 to about 6.00, about 1.05 to about 5.50, about 1.05 to about 5.00, about 1.05 to about 4.50, about 1.05 to about 4.00, about 1.05 to about 3.80, about 1.05 to about 3.60, about 1.05 to about 3.40, about 1.05 to about 3.20, about 1.05 to about 3.00, about 1.05 to about 2.80, about 1.05 to about 2.60, about 1.05 to about 2.40, about 1.05 to about 2.20, about 1.05 to about 2.00, about 1.05 to about 1.90, about 1.05 to about 1.80, about 1.05 to about 1.70, about 1.05 to about 1.60, about 1.05 to about 1.50, about 1.05 to about 1.40, about 1.05 to about 1.30, about 1.05 to about 1.20, or about 1.05 to about 1.10. For example, the number of long identified neoantigen peptides could be 150 for union sequencing results and 100 for an initial sequencing result of a biological sample (i.e., a ratio of about 1.50), indicating the scoring of predicted immunogenicity of one or more neoantigens will be based on the union sequencing results.

### Neoantigens

Neoantigens are self-antigens generated by tumor cells due to genomic mutations or dysregulated RNA splicing. Neoantigens identified by sequencing in methods described herein can be in the form of any sequencing results including, but not limited to, sequence reads (DNA sequence reads, RNA sequence reads), sequence variants (e.g., peptide-modifying sequence variants), encoded peptides, or combinations thereof. The neoantigens can be any type of peptide, including but not limited to long peptides, short peptides, or combinations thereof. In some embodiments, the one or more neoantigens are peptides that are long peptides, short peptides, or a combination thereof. Neoantigens of methods described herein can be tumor-specific (e.g., neoantigens only present in a cancer or tumor and not in healthy or germ-line cells of a subject).

### Scoring the Predicted Immunogenicity of a Neoantigen

Sequencing results obtained by methods described herein can be used to determine peptide sequences that are encoded by nucleic acids (e.g., encoded by DNA, encoded by RNA). The resulting peptide sequences can be analyzed to determine whether the encoded peptide is a neoantigen based on the presence of sequence variants (e.g., sequence variants in the peptide amino acid sequence compared to the healthy cells or healthy tissue of a subject, sequence variants in the peptide amino acid sequence compared to a reference genome). Neoantigens (e.g., neoantigen peptides) identified by methods described herein can be analyzed to determine a predicted immunogenicity based on any factor including, but not limited to whether the neoantigen is immunogenic (e.g., whether a neoantigen can elicit an immune response in a subject, prediction of major histocompatibility complex (MHC) binding affinity, whether peptide is predicted to be presented on a cell surface by an MHC molecule), whether the tumor expresses an amount of neoantigen sufficient to elicit an immune response, whether the neoantigen is expressed on a sufficient fraction of the tumor cells, or a combination thereof. Any methodology for predicting immunogenicity of a neoantigen can be used in methods described herein. For example, the number of sequenced variants (e.g., DNA sequence variants, RNA sequence variants, encoded peptide variants) can be used to predict the expression of a neoantigen in a biological sample and infer the expression in the originating tissue of the subject (e.g., in a tumor of the subject). For several examples of predicting immunogenicity of a neoantigen, see WO2022/159176 A1, US 20230197192A1, US 20230173045A1, and US 20220093209A1, all of which are hereby incorporated by reference in their entireties. The predicted immunogenicity can be used to score a neoantigen. The scores of predicted immunogenicity can be ranked (e.g., neoantigens can be ranked in order of predicted immunogenicity). The scored neoantigens can be further selected for inclusion (e.g., inclusion of a neoantigen peptide, inclusion of a nucleic acid encoding for a neoantigen peptide) in a neoantigen vaccine. The scored neoantigens can be further selected for exclusion (e.g., exclusion of a neoantigen peptide, exclusion of a nucleic acid encoding for a neoantigen peptide) in a neoantigen vaccine.

### Neoantigen Vaccines and Methods of Administration

Methods described herein can include the step of generating a neoantigen vaccine. The neoantigen vaccine can be any immunogenic composition, such as those described in WO2022/170067 A1, US2023/0173045 A1, WO2022/159176 A1, WO2022/251034 A1, or US2023/0173046 A1, the entirety of all of which are incorporated by reference herein. The neoantigen vaccine can comprise or encode neoantigens (e.g., one or more neoantigens) scored for predicted immunogenicity by the methods described herein. The neoantigen vaccine can contain neoantigens in any form, including but not limited to peptides (e.g., long identified peptides, short identified peptides, synthesized peptides, isolated peptides), RNA sequences encoding for peptides (e.g., mRNA, mRNA containing unnatural nucleotides (e.g., pseudouridine, N1-methylpseudouridine, 7-methylguanosine, N6-methyladenosine, 2′-O-methyl nucleotide), mRNA containing inverted nucleotides, or combinations thereof), DNA sequences encoding for peptides (e.g., plasmid DNA, viral DNA), or combinations thereof. Neoantigen vaccines containing nucleic acids (e.g., RNA, DNA) encoding for peptides can be in the form of a virus (e.g., an adenovirus, lentivirus, fowl pox, vaccinia, self-replicating alphavirus, Maraba virus).

A nucleic acid (e.g., RNA, DNA) of a neoantigen vaccine can encode for any number of peptide antigens. The number of peptide antigens encoded by a nucleic acid can be about 1, about 2, about 3, about 4, about 5, about 6, about 7, about 8, about 9, about 10, about 11, about 12, about 13, about 14, about 15, about 16, about 17, about 18, about 19, or about 20. The number of peptide antigens encoded by a nucleic acid can be at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20. In some embodiments, the method includes the step of generating a neoantigen vaccine, wherein the neoantigen vaccine comprises or encodes for one or more neoantigens scored for the predicted immunogenicity by the method.

A neoantigen vaccine can contain or encode for any number of neoantigens (e.g., neoantigen proteins, DNA sequences encoding for neoantigen proteins, RNA sequences encoding for neoantigen proteins). Neoantigen vaccines can contain or encode for a number of neoantigens that is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30. The number of neoantigens contained or encoded by a neoantigen vaccine can be one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, or 29 or more, or 30 or more. The number of neoantigens contained or encoded by a neoantigen vaccine can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30. The number of neoantigens contained or encoded by a neoantigen vaccine can be between about 1 to about 50, about 2 to about 50, about 3 to about 50, about 4 to about 50, about 5 to about 50, about 6 to about 50, about 7 to about 50, about 8 to about 50, about 9 to about 50, about 10 to about 50, about 11 to about 50, about 12 to about 50, about 13 to about 50, about 14 to about 50, about 15 to about 50, about 16 to about 50, about 17 to about 50, about 18 to about 50, about 19 to about 50, about 20 to about 50, about 22 to about 50, about 24 to about 50, about 26 to about 50, about 28 to about 50, about 30 to about 50, about 33 to about 50, about 36 to about 50, about 40 to about 50, about 45 to about 50, about 1 to about 45, about 1 to about 40, about 1 to about 36, about 1 to about 33, about 1 to about 30, about 1 to about 28, about 1 to about 26, about 1 to about 24, about 1 to about 22, about 1 to about 20, about 1 to about 18, about 1 to about 16, about 1 to about 14, about 1 to about 12, about 1 to about 10, about 1 to about 9, about 1 to about 8, about 1 to about 7, about 1 to about 6, about 1 to about 5, about 1 to about 4, about 1 to about 3, about 1 to about 2, about 2 to about 14, about 2 to about 12, about 2 to about 10, about 2 to about 8, about 2 to about 6, about 2 to about 4, about 3 to about 14, about 3 to about 12, about 3 to about 10, about 3 to about 8, about 3 to about 6, about 3 to about 4, about 4 to about 14, about 4 to about 12, about 4 to about 10, about 4 to about 8, about 4 to about 6, about 5 to about 14, about 5 to about 12, about 5 to about 10, about 5 to about 8, about 5 to about 6, about 6 to about 14, about 6 to about 12, about 6 to about 10, about 6 to about 8, about 7 to about 14, about 7 to about 12, about 7 to about 10, about 7 to about 8, about 8 to about 14, about 8 to about 12, about 8 to about 10, about 9 to about 14, about 9 to about 12, about 9 to about 10, about 10 to about 14, about 10 to about 12, about 11 to about 14, about 11 to about 12, about 12 to about 14, or about 13 to about 14.

Neoantigen vaccines described herein may comprise up to about 50 neoantigen long peptides and/or short peptides. The Neoantigen vaccine may comprise about 10 to about 20 neoantigen long peptides and/or short peptides. In some embodiments, the neoantigen vaccine comprises about 19 neoantigen long peptides and/or short peptides.

The neoantigen vaccine may comprise at least about 2 or more neoantigen long peptides. The neoantigen vaccine may comprise about 2 to about 18 neoantigen long peptides. The neoantigen vaccine can comprise at least about 10 to about 15 neoantigen long peptides. The neoantigen vaccine may comprise at least about 2 or more neoantigen short peptides. The neoantigen vaccine may comprise at least about 2 to about 10 neoantigen short peptides.

As an example, three peptide pools of the neoantigen vaccine may comprise about 5 neoantigen long peptides and/or short peptides and one peptide pool may comprise 4 neoantigen long peptides and/or short peptides and a helper peptide. Each peptide pool may comprise different neoantigen long peptides and/or short peptides. Neoantigen long peptides may be about 15 to about 30 amino acids in length. Neoantigen short peptides may be about 5 to about 15 amino acids in length.

The neoantigen vaccine can comprise at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or more neoantigen peptides (e.g., neoantigen long peptides and/or short peptides). The neoantigen vaccine can comprise up to about 100 neoantigen peptides. The neoantigen vaccine can contain about 10-20 neoantigens, about 10-30 neoantigens, about 10-40 neoantigens, about 10-50 neoantigens, about 10-60 neoantigens, about 10-70 neoantigens, about 10-80 neoantigens, about 10-90 neoantigens, or about 10-100 neoantigens. In some embodiments, the neoantigen vaccine comprises at least about 10 neoantigens. In some embodiments, the neoantigen vaccine disclosed herein can comprises 10 to about 20 neoantigens. For example, the neoantigen vaccine can comprise about 10, about 11, about 12, about 13, about 14, about 15, about 16, about 17, about 18, about 19, or about 20 neoantigens. In some embodiments, the neoantigen vaccine can comprise about 19 neoantigens. In some embodiments, neoantigen vaccine can comprise about 20 neoantigens. Each of the neoantigens in the neoantigen vaccine can be different.

The neoantigen vaccines described herein can further comprise an adjuvant. Adjuvants are any substance whose admixture into a neoantigen vaccine increases, or otherwise enhances and/or boosts, the immune response to a tumor-specific neoantigen, but when the substance is administered alone does not generate an immune response to a tumor-specific neoantigen. The adjuvant can generate an immune response to the neoantigen and does not produce an allergy or other adverse reaction. It is contemplated herein that adjuvant can be administered before, together, concomitantly with, or after administration of the neoantigen vaccine.

Adjuvants can enhance an immune response by several mechanisms including, e.g., lymphocyte recruitment, stimulation of B and/or T cells, and stimulation of macrophages. When a neoantigen vaccine described herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, immunostimulatory adjuvants, or combinations thereof. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), AS03 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), poly ICLC, non-CpG bacterial DNA or RNA as well as immunoactive small molecules and antibodies such as cyclophosphamide, sunitmib, bevacizumab, Celebrex (celecoxib), NCX-4016, sildenafil, tadalafil, vardenafil, sorafenib, XL-999, CP-547632, pazopanib, ZD2171, AZD2171, ipilimumab, tremelimumab, and SC58175, which may act therapeutically and/or as an adjuvant.

Neoantigen vaccines described herein can be administered to a subject that has been diagnosed with cancer, is already suffering from cancer, has recurrent cancer (i.e., relapse), or is at risk of developing cancer. A neoantigen vaccine can be administered to a subject that is resistant to other forms of cancer treatment (e.g., chemotherapy, immunotherapy, or radiation). Neoantigen vaccines can be administered to the subject prior to other standard of care cancer therapies (e.g., chemotherapy, immunotherapy, or radiation). Neoantigen vaccines can be administered to the subject concurrently, after, or in combination to other standard of care cancer therapies (e.g., chemotherapy, immunotherapy, or radiation). In some embodiments, the method includes the step of administering the neoantigen vaccine to a subject in need thereof.

Neoantigen vaccines described herein can be administered to a subject in an amount sufficient to elicit an immune response to the tumor-specific neoantigen and to destroy, or at least partially arrest, symptoms and/or complications. In embodiments, the neoantigen vaccine can provide a long-lasting immune response. A long-lasting immune response can be established by administering a boosting dose of the neoantigen vaccine to the subject. The immune response to the neoantigen vaccine can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that neoantigen vaccines can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of neoantigen vaccines.

A neoantigen vaccine described herein can be administered to the subject alone or in combination with other therapeutic agents. The therapeutic agent can be, for example, a chemotherapeutic agent, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochlorperazine, rituximab, tamoxifen, topotecan hydrochloride, trastuzumab, vinblastine, vincristine and vinorelbine tartrate. The subject may be administered a small molecule, or targeted therapy (e.g., kinase inhibitor). The subject may be further administered an anti-CTLA antibody or anti-PD-1 antibody or anti-PD-L1 antibody. Blockade of CTLA-4 or PD-L1 by antibodies can enhance the immune response to cancerous cells in the patient.

### EXAMPLES

### Example 1. Analysis of Initial Sequencing Results and Union Sequencing Results of Patient Tissue Biopsies

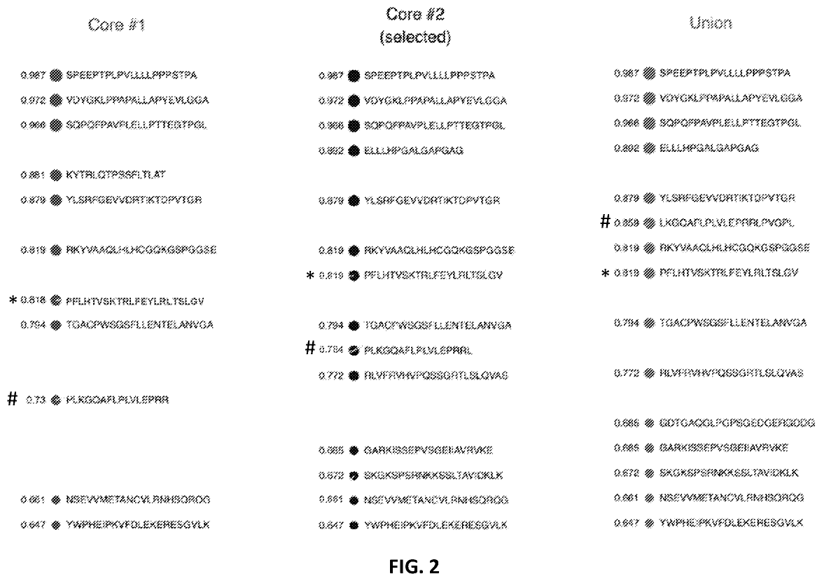

Core needle biopsies (i.e., Core #1 and Core #2) were collected from a tumor of patient A and processed for nucleic acid sequencing by whole exome sequencing (WES). The raw sequencing reads of each core needle biopsy were analyzed for encoded neoantigen peptides. The observed neoantigen peptides were scored for predicted immunogenicity and ranked by predicted immunogenicity score (see FIG. 2, left and middle columns). The scoring of predicted immunogenicity was conducted an analogous fashion to methods described in US 20230197192A1. The raw sequencing reads of both core needle biopsy samples (e.g., both WES DNA and bulk RNA sequencing reads) were concatenated and analyzed for encoded neoantigen peptides (e.g., the union sequencing results). The observed neoantigen peptides of the union sequencing results were scored for predicted immunogenicity and ranked by predicted immunogenicity analogously to the individual core needle biopsy cores (see FIG. 2, right column). Exemplary peptides that are overlapping or encompassed by each other are denoted by and “#” symbols in FIG. 2. Of note, the union sequencing result identified a neoantigen peptide

(SEQ ID NO: XXX)
(LKGQAFLPLVLEPRRLPVGPL,

score of 0.859) with a higher predicted immunogenicity score than in either of the cores (denoted with “#”, Core #1:

(SEQ ID NO: XXX)
PLKGQAFLPLVLEPRR,
Core #2:
(SEQ ID NO: XXX)
PLKGQAFLPLVLEPRRL

wherein underline indicates the amino acids in overlap, score of 0.73 and 0.784, respectively). Analogous sequencing and analyses were conducted for separate core needle biopsy samples from a different patient (i.e., patient B, results not shown).

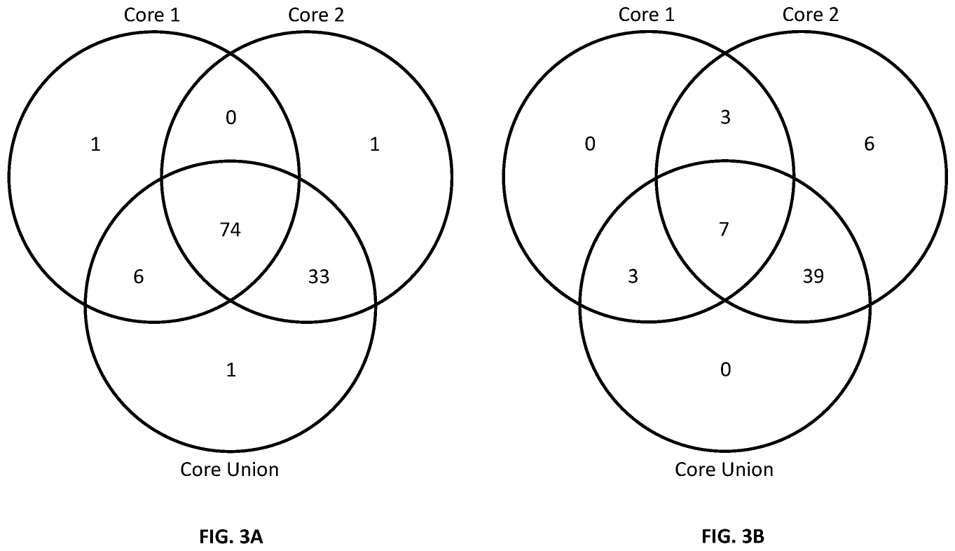

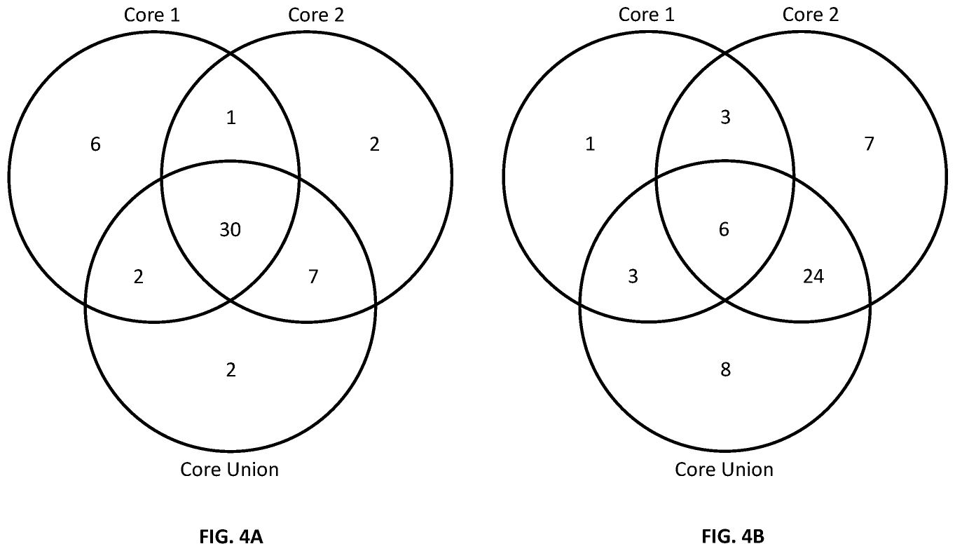

The number of long identified neoantigen peptides from patient A and patient B were visualized by Venn diagrams of each core needle biopsy sequencing result and each union sequencing result (see FIGS. 3A and 3B, respectively). Results from patient A indicated that each of the core 1, core 2, and union sequencing results contained only one exclusive long identified neoantigen peptide not present in other sequencing results (see FIG. 3A). In contrast, the sequencing results from patient B indicated that core 2 contained six exclusive long identified neoantigen peptides (see FIG. 3B). By using a cutoff of the top 48 ranking peptides by predicted immunogenicity, samples were selected for potential inclusion in a neoantigen vaccine. Venn diagrams using such a cutoff are shown in FIGS. 4A and 4B, corresponding to a subset of total long identified neoantigen peptide Venn diagrams in FIGS. 3A and 3B, respectively.

### Example 2. Analysis of Sequencing Parameters from the Initial Nucleic Acid Sequencing Results of Patient Tissue Samples

Two core needle biopsy samples were collected from each of three patients (i.e., patients C, D, and E) and prepared for whole exome sequencing of DNA. The sequencing parameters of each initial sequencing result are displayed in Table 1. In some instances (e.g., total variants for patient D, number of long identified neoantigen peptides or RNA confirmed protein-modifying variants for patient E), the sequencing parameter values were nearly equivalent between the two core needle biopsy samples of a particular patient (e.g., a ratio of sequencing parameter values was approximately equivalent to 1.0). In other occasions (e.g., number of protein-modifying variants or RNA confirmation rate of patient C), the sequencing parameter values were not equivalent between the two core needle biopsy samples of a particular patient (e.g., a ratio of sequencing parameter values is not about 1.0, a ratio of sequencing parameter values of at least 1.5). Table 1 shows the sample selected as a representative biological sample from a group of biological samples.

A neoantigen vaccine, containing identified neoantigen peptides was generated for each patient. The neoantigen vaccine for patient D was based on the predicted immunogenicity scores of the combination of cores 1 and 2 (i.e., union sequencing results). The neoantigen vaccine for patient C and patient E were based on the predicted immunogenicity scores of representative cores 1 and 2, respectively. The decision of which core to select as the representative core for each patient, or whether to combine sequencing results of cores 1 and 2 was based on the sequencing parameters of Table 1 and generated Venn diagrams of identified neoantigen peptides (similar to those shown in FIGS. 3A, 3B, 4A, and 4B; i.e., the number of identified neoantigen peptides in each sequencing result, shared between sequencing results, or unique to each sequencing result).

TABLE 1
Sequencing parameters of patient C,
D, and E initial sequencing results

| | Patient | | | | | |
|---|---|---|---|---|---|---|
| | C | | D | | E | |
| | WES Core | | | | | |
| | Core | Core | Core | Core | Core | Core |
| | 1 | 2 | 1 | 2 | 1 | 2 |
| Tumor | 0.87 | 0.5 | 0.5 | 0.68 | 0.63 | 0.65 |
| Purity | | | | | | |
| Sequencing | 196 x | 277 x | 273 x | 206 x | 190 x | 286 x |
| Depth | | | | | | |
| Total | 352 | 87 | 145 | 130 | 3,993 | 4,008 |
| Variants | | | | | | |
| Protein- | 125 | 38 | 61 | 51 | 1,335 | 1,342 |
| modifying | | | | | | |
| Variants | | | | | | |
| RNA | 36 | 0 | 16 | 22 | 393 | 396 |
| Confirmed, | | | | | | |
| Protein- | | | | | | |
| modifying | | | | | | |
| Variants | | | | | | |
| RNA | 28.8 | 0 | 26.2 | 43.1 | 29.4 | 29.5 |
| Confirmation | | | | | | |
| Rate (%) | | | | | | |
| Long | 35 | 0 | 0 | 24 | 38 | 396 |
| Identified | | | | | | |
| Neoantigen | | | | | | |
| Peptides | | | | | | |
| Short | 250 | 0 | 106 | 213 | 2579 | 2621 |
| Identified | | | | | | |
| Neoantigen | | | | | | |
| Peptides | | | | | | |
| Selection? | Y | N | N | Y | N | Y |
| (Y/N) | | | | | | |

What is claimed is:

1. A method of scoring a predicted immunogenicity of one or more neoantigens in one or more biological samples of a subject, comprising the steps of
a) preparing two or more biological samples for nucleic acid sequencing;
b) nucleic acid sequencing of the two or more biological samples to yield initial sequencing results for each of the biological samples;
c) evaluating the initial sequencing results by analyzing one or more sequencing parameters of the initial sequencing results;
d) based on an analysis of the sequencing parameters of the initial sequencing results of the biological samples, combining the initial sequencing results of the two or more biological samples to yield union sequencing results; and
e) scoring the predicted immunogenicity of one or more neoantigens in the biological samples of the subject in need thereof based on the union sequencing results of the biological samples.

2. A method of scoring a predicted immunogenicity of one or more neoantigens in one or more biological samples of a subject, comprising the steps of
a) preparing two or more biological samples for nucleic acid sequencing;
b) nucleic acid sequencing of the two or more biological samples to yield initial sequencing results for each of the biological samples;
c) evaluating the initial sequencing results by analyzing one or more sequencing parameters of the initial sequencing results;
d) based on an analysis of the sequencing parameters of the initial sequencing results of the biological samples, selecting a representative biological sample;
e) scoring the predicted immunogenicity of one or more neoantigens in the representative biological sample of the subject in need thereof based on the sequencing result of the representative biological sample.

3. The method of claim 1, wherein each of the biological samples is independently a biopsy sample selected from the group consisting of an excisional biopsy, a liquid biopsy, an incisional biopsy, a needle biopsy, a punch biopsy, and a shave biopsy.

4. The method of claim 1, wherein at least two of the biological samples are from different regions of the body of the subject.

5. The method of claim 1, wherein at least two of the biological samples are from different regions of a tumor of the subject.

6. The method of claim 1, wherein at least one of the biological samples is from a primary tumor of the subject.

7. The method of claim 1, wherein at least one of the biological samples is from a secondary tumor of the subject.

8. The method of claim 1, wherein at least two of the biological samples are collected from the subject at different occasions that are temporally separated by at least about 1 day.

9. The method of claim 1, wherein the sequencing parameters are selected from the group consisting of a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, and combinations thereof.

10. The method of claim 1, wherein the initial sequencing results are selected from the group consisting of sequencing reads, sequencing variants, encoded peptides, and combinations thereof.

11. The method of claim 1, further comprising the step of comparing one or more union sequencing parameters of the union sequencing results to the one or more sequence parameters of the initial sequencing results of step c).

12. The method of claim 11, wherein the union sequencing parameters are selected from the group consisting of a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, and combinations thereof.

13. The method of claim 1, wherein the nucleic acid sequencing is sequencing of a nucleic acid that is RNA, DNA, or a combination thereof.

14. The method of claim 1, wherein the nucleic acid sequencing is selected from the group consisting of whole exome sequencing, whole genome sequencing, RNA sequencing, single cell sequencing, targeted panel sequencing, and combinations thereof.

15. The method of claim 1, wherein at least two different sequencing techniques are used in the step of nucleic acid sequencing of the biological samples.

16. The method of claim 1, wherein at least two of the biological samples are sequenced by different sequencing techniques in the step of nucleic acid sequencing.

17. The method of claim 1, further comprising the step of subsequent sequencing of the biological samples.

18. The method of claim 17, wherein the subsequent sequencing is selected from the group consisting of whole exome sequencing, whole genome sequencing, RNA sequencing, single cell sequencing, targeted panel sequencing, and combinations thereof.

19. The method of claim 17, wherein at least two of the biological samples are sequenced by different sequencing techniques in the step of subsequent sequencing.

20. The method of claim 1, wherein the one or more neoantigens are peptides that are selected from the group consisting of long peptides, short peptides, and a combination thereof.

21. The method of claim 1, further comprising the step of generating a neoantigen vaccine, wherein the neoantigen vaccine comprises or encodes for one or more neoantigens scored for the predicted immunogenicity.

22. The method of claim 21, further comprising the step of administering the neoantigen vaccine to the subject in need thereof.

23. The method of claim 2, wherein the sequencing parameters are selected from the group consisting of a number of sequence reads, a number of sequencing variants, a tumor purity, a sequencing depth, a number of protein modifying sequencing variants, an RNA confirmation rate, an RNA quality, a number of long identified neoantigen peptides, a number of short identified neoantigen peptides, a predicted immunogenicity of a neoantigen peptide, and combinations thereof.

24. The method of claim 2, wherein the nucleic acid sequencing is selected from the group consisting of whole exome sequencing, whole genome sequencing, RNA sequencing, single cell sequencing, targeted panel sequencing, and combinations thereof.

25. The method of claim 2, further comprising the step of subsequent sequencing of the representative biological sample.

26. The method of claim 25, wherein the subsequent sequencing is selected from the group consisting of whole exome sequencing, whole genome sequencing, RNA sequencing, single cell sequencing, targeted panel sequencing, and combinations thereof.
